(19) United States
(12) Patent Application Publication
(10) Pub. No.: US 2016/0340737 A1
(43) Pub. Date: Nov. 24, 2016

(54) EPIGENETIC STEM CELL COMMITMENT-ASSOCIATED SIGNATURE

(71) Applicant: ALBERT EINSTEIN COLLEGE OF MEDICINE, INC., Bronx, NY (US)

(72) Inventors: Ulrich G. Steidl, New Rochelle, NY (US); Amit Verma, Bronxville, NY (US); Boris Bartholdy, Bronx, NY (US); Maximilian Christopeit, New York, NY (US)

(73) Assignee: Albert Einstein College of Medicine, Inc., Bronx, NY (US)

(21) Appl. No.: 15/111,869

(22) PCT Filed: Dec. 29, 2014
(86) PCT No.: PCT/US14/72474

§ 371 Date: Jul. 15, 2016

Related U.S. Application Data
(60) Provisional application No. 61/932,973, filed on Jan. 29, 2014.

(51) Int. Cl. C12Q 1/68; A61K 31/704; A61K 35/545

(52) U.S. Cl. CPC C12Q 1/6886; A61K 35/545; A61K 31/704; C12Q 2600/154; C12Q 2600/118

(57) ABSTRACT

Methods for determining the prognosis of a subject having acute myeloid leukemia (AML) as well as methods of treating AML subjects depending on prognosis.

### CROSS-REFERENCE TO RELATED APPLICATIONS

This application claims benefit of U.S. Provisional Application No. 61/932,973, filed Jan. 29, 2014, the contents of which are hereby incorporated by reference.

### STATEMENT OF GOVERNMENT SUPPORT

This invention was made with government support under grant number R00CA131503 awarded by the National Cancer Institute. The government has certain rights in the invention.

### BACKGROUND OF THE INVENTION

The disclosures of all patents, patent application publications and publications referred to in this application, including those cited to by number in parentheses, are hereby incorporated by reference in their entirety into the subject application to more fully describe the art to which the subject invention pertains.

In the pathogenesis of acute myeloid leukemia (AML), genes encoding epigenetic modifiers are frequently mutated (1, 2). Some of these mutations have been attributed prognostic value in AML (3). Additionally, aberrant DNA cytosine methylation in AML blasts has led to the identification of novel AML subtypes, independent of features usually associated with AML (4). Differentiation of murine HSC to progenitor cells is associated with distinct changes in DNA cytosine methylation (5-7). In turn, targeted disruption of DNA cytosine methylation patterns disturbs regulation of differentiation of murine hematopoietic stem and progenitor cells (HSPC), and affects HSPC function (8-10). This suggests that methylation plays an active role in the differentiation program.

In the murine hematopoietic system, dynamic changes of DNA methylation have been described during multipotent progenitor cell differentiation (5) and hematopoietic stem cell commitment (7), with pronounced demethylation in erythroid progenitors during differentiation (6, 7). Severely perturbed hematopoiesis (8-11) and myeloid transformation (12-14) are common hallmarks of mouse models with targeted disruptions in a growing number of enzymes known to contribute to the homeostasis of DNA cytosine methylation. However, little is known about changes in DNA cytosine methylation during early human hematopoiesis. Identification of stage- and locus-specific epigenetic mechanisms of leukemic transformation would require a detailed genome wide map of DNA cytosine methylation patterns and dynamics during the step-wise maturation of hematopoietic stem cells (HSC). Currently there are no identified stage-specific and locus-specific epigenetic mechanisms of leukemic transformation.

The present invention provides a stem cell commitment-associated methylome that is independently prognostic of poorer overall survival in AML.

### SUMMARY OF THE INVENTION

This invention provides a method for determining the prognosis of a subject having acute myeloid leukemia (AML), comprising
a) quantifying the methylation of DNA of a sample comprising blood cells or bone marrow cells obtained from a subject with AML at a plurality of chromosome loci, or nearest associated gene, as listed in Table 2;
b) determining a methylation score from the methylation determined in step a);
c) comparing the methylation score of DNA of the sample of blood cells obtained from the subject with AML with a predetermined reference amount for the same plurality of chromosome loci, or nearest associated gene, and
d) assigning a prognosis to the subject,
wherein a methylation score at or in excess of the predetermined reference amount indicates a negative prognosis for the subject,
and wherein a methylation score below the predetermined reference amount indicates a positive prognosis for the subject.

Also provided is a method for determining the prognosis of a subject having acute myeloid leukemia (AML), comprising
quantifying methylation of DNA of a sample comprising blood cells obtained from a subject with AML at a plurality of the chromosome loci, or nearest associated gene, listed in Table 2 as demethylated at STHSC-CMP transition and/or at a plurality of the chromosome loci, or nearest associated gene, listed in Table 2 as methylated at STHSC-CMP transition,
comparing the extent of the methylation with the methylation of DNA at the same plurality or pluralities of chromosome loci, or nearest associated gene, in a sample of blood cells obtained from a subject without AML, and
assigning a prognosis to the subject,
wherein demethylation of the majority of the plurality of loci or nearest associated gene listed in Table 2 as demethylated at STHSC-CMP transition in the DNA of the sample from the AML subject compared to the DNA of the sample of the subject without AML indicates a negative prognosis for the subject, and wherein methylation of the majority of the plurality of loci or nearest associated gene listed in Table 2 as methylated at STHSC-CMP transition in the DNA of the sample from the AML subject compared to the DNA of the sample from the subject without AML indicates a negative prognosis for the subject,
and wherein demethylation of the minority of the plurality of loci or nearest associated gene listed in Table 2 as demethylated at STHSC-CMP transition in the DNA of the sample from the AML subject compared to the DNA of the sample of the subject without AML indicates a negative prognosis for the subject, and wherein methylation of the minority of the plurality of loci or nearest associated gene listed in Table 2 as methylated at STHSC-CMP transition in the DNA of the sample from the AML subject compared to the DNA of the sample from the subject without AML indicates a negative prognosis for the subject.

Also provided is a method for treating a subject having acute myeloid leukemia (AML) comprising determining the prognosis of the subject, by any of the methods described herein, as positive or negative, and treating the subject with a chemotherapy if the subject has a positive prognosis or treating the subject with a non-chemotherapeutic method if the subject has a negative prognosis.

Also provided is a microarray comprising a nucleic acid probe for all, or for less than all, of the 561 loci or nearest associated genes listed in Table 2.

Also provided is a kit for determining the prognosis of a subject having acute myeloid leukemia (AML), comprising
a) reagents for quantifying the methylation of DNA of a sample comprising blood cells or bone marrow cells obtained from a subject with AML at a plurality of chromosome loci, or nearest associated gene, as listed in Table 2;
b) written instructions for determining a methylation score from the methylation determined with the reagents in a);
c) written instructions for a predetermined reference amount for the same plurality of chromosome loci, or nearest associated gene, and for assigning a prognosis to the subject based on the methylation score compared to the predetermined reference amount,
wherein a methylation score at or in excess of the predetermined reference amount indicates a negative prognosis for the subject,
and wherein a methylation score below the predetermined reference amount indicates a positive prognosis for the subject.

### BRIEF DESCRIPTION OF THE DRAWINGS

FIG. 1A-1D. Hypomethylation during HSC commitment to hematopoietic progenitors—A: Genome-wide changes in DNA methylation during HSC commitment. Red dots represent loci with significantly lower methylation at the developmentally later stage, i.e. loci demethylated during the respective transition (p<0.05, t-test). B: Significant changes in DNA cytosine methylation at the transition from LTHSC to STHSC (outer circle), STHSC to CMP (middle circle), and CMP to MEP (inner circle) (LTHSC=long-term HSC; STHSC=short-term HSC; CMP=common myeloid progenitors; MEP=megakaryocyte-erythrocyte progenitors) are plotted in relation to the genomic position. Chromosomes are plotted along the ideogram. Red bars denote significantly demethylated loci, green bars denote significant increase in methylation at the respective locus. C: SAM was used to define an epigenetic signature based on loci which undergo the most significant methylation changes during HSPC differentiation. The epigenetic signature (561 loci) distinguishes HSPC subsets in hierarchical clustering analysis. Log 2-transformed HpaII/MspI ratios (color code next to the heat map) of all 561 loci are shown for the four analyzed differentiation stages (indicated above the heat map) from three healthy human individuals. Trees result from Euclidean clustering of this signature. Associated genes are listed in Table 2. D: Ingenuity pathway analysis highlights functional implications of gene enrichment analysis of the epigenetic stem cell commitment-associated signature. 62 genes significantly associated (p<0.05 after Benjamini-Hochberg-correction) with Ingenuity Top Bio Functions were entered into pathway generation. Top five canonical pathways (“AML Signaling”, “Molecular Mechanisms of Cancer”, “Glioblastoma Multiforme Signaling”, “Pancreatic Adenocarcinoma Signaling”, “Glucocorticoid Receptor Signaling”) and the top three characteristics of function and disease (“Differentiation of Blood Cells” (p=8.39E-43), “Lymphohematopoietic Cancer” (p=3.2E-12), “AML” (p=2.47E-06), and “Cell Transformation” (p=1.41E-12)) are depicted.

FIG. 2A-2H. Stem cell commitment-associated epigenetic signature is prognostic for overall survival in AML—Application of the epigenetic signature to three independent published sets of patients with AML (4, 39-42). A, B: Analysis of patients with AML who received standard chemotherapy. C, D: Analysis of patients with AML who received chemotherapy with a higher dose of daunorubicin (DNR). E: Analysis of the combined cohort of AML patients receiving standard or higher doses of daunorubicin (41). H: Analysis of a third independent cohort of AML patients (39, 40). A, C, G: Heat map of the respective patients (horizontal order) and the 561 loci (vertical order). Patients are ranked in descending order based on the signature score. The score was calculated by summing absolute values of the median-centered methylation values (log 2[HpaII/MspI]) of the 561 signature loci for each patient sample. Patients with high signature score are indicated by a green bar, patients with a low signature score by a black bar above the median-centered methylation heat map. B, D, E: Kaplan-Meier survival curves of OS of patients with AML are plotted. Green solid lines represent OS of patients with a high signature score, black solid lines represent OS of patients with a low signature score. F: Overlay of survival curves from FIG. 2B, D. Black/red lines: patients with a low epigenetic stem cell commitment-associated signature score receiving standard or high dose daunorubicin treatment. Green/blue lines: patients with a high epigenetic stem cell commitment-associated signature score receiving standard or high dose daunorubicin treatment.

FIG. 3A-3D. Lower prognostic power of gene expression signature—A: Generation of a gene expression signature based on 455 gene expression probes that undergo significant changes between the four measured differentiation stages using SAM. Heat map of log 2-transformed expression values of this signature is shown. B-E: Application of the stem cell commitment-associated gene expression signature to patients with AML. B, D: Heat maps of median-centered expression of the probes corresponding to the commitment-associated gene expression signature in patients with AML who received standard chemotherapy (B), or chemotherapy with a higher dose of daunorubicin (D). C, E: Kaplan-Meier curves of OS of patients with AML are plotted; green lines represent OS of patients with a high expression signature score, black lines represent OS of patients with a low expression signature score.

FIG. 4. Correlation of epigenetic signature's constituents with expression of closest mappable gene product—Changes of the 561 constituents of the epigenetic signature during transition from STHSC to CMP are aligned with significant changes in gene expression at mappable loci nearby. Red represents demethylated loci, green methylated loci during STHSC to CMP transition, yellow represents increased and blue decreased gene expression at associated loci.

FIG. 5. Correlation between methylation and expression of genes in the commitment-associated signatures—Changes in DNA cytosine methylation and gene expression between the indicated differentiation stages were calculated (mean methylation in later vs. earlier compartment, and mean expression in later vs. earlier compartment) and plotted in the graphs. The black line represents the linear model of these data points, and the p-value for correlation is indicated. A: Using the stem cell commitment-associated 561 probe epigenetic signature, 530 pairs of methylation probe with adjacent transcript were mapped and their correlation is shown. B: Similarly, the commitment-associated 455 probe expression signature was used to derive 283 pairs of transcripts associated with nearby or overlapping methylation probes, which are plotted in the figure.

### DETAILED DESCRIPTION OF THE INVENTION

This invention provides a method for determining the prognosis of a subject having acute myeloid leukemia (AML), comprising
a) quantifying the methylation of DNA of a sample comprising blood cells or bone marrow cells obtained from a subject with AML at a plurality of chromosome loci, or nearest associated gene, as listed in Table 2;
b) determining a methylation score from the methylation determined in step a);
c) comparing the methylation score of DNA of the sample of blood cells obtained from the subject with AML with a predetermined reference amount for the same plurality of chromosome loci, or nearest associated gene, and
d) assigning a prognosis to the subject,
wherein a methylation score at or in excess of the predetermined reference amount indicates a negative prognosis for the subject,
and wherein a methylation score below the predetermined reference amount indicates a positive prognosis for the subject.

In an embodiment, the sample comprises blood cells. In an embodiment, the sample comprises bone marrow cells.

In an embodiment, the methylation score comprises a direct or indirect measurement of the ratio of demethylated CpG residues/methylated+demethylated CpG residues of the plurality of the chromosome loci, or nearest associated gene for the DNA of a sample.

In an embodiment, the methylation is determined by a isoschizomer enzyme pair method, and wherein the methylation score is obtained by summing absolute values of the median-centered methylation values (log 2[methylation sensitive enzyme measured fragments/methylation insensitive enzyme measured fragments]) of the plurality of the chromosome loci, or nearest associated gene for the DNA of a sample.

In an embodiment, the isoschizomer enzyme pair is HpaII and MspI.

In an embodiment, the HELP assay is used to determine the methylation of the DNA.

In one embodiment, the blood or bone marrow sample has previously been obtained from the subject. Also provided is a method as described hereinabove but further comprising the step of obtaining the blood or bone marrow sample from the subject.

Also provided is a method for determining the prognosis of a subject having acute myeloid leukemia (AML), comprising
quantifying methylation of DNA of a sample comprising blood cells obtained from a subject with AML at a plurality of the chromosome loci, or nearest associated gene, listed in Table 2 as demethylated at STHSC-CMP transition and/or at a plurality of the chromosome loci, or nearest associated gene, listed in Table 2 as methylated at STHSC-CMP transition,
comparing the extent of the methylation with the methylation of DNA at the same plurality or pluralities of chromosome loci, or nearest associated gene, in a sample of blood cells obtained from a subject without AML, and
assigning a prognosis to the subject,
wherein demethylation of the majority of the plurality of loci or nearest associated gene listed in Table 2 as demethylated at STHSC-CMP transition in the DNA of the sample from the AML subject compared to the DNA of the sample of the subject without AML indicates a negative prognosis for the subject, and wherein methylation of the majority of the plurality of loci or nearest associated gene listed in Table 2 as methylated at STHSC-CMP transition in the DNA of the sample from the AML subject compared to the DNA of the sample from the subject without AML indicates a negative prognosis for the subject,
and wherein demethylation of the minority of the plurality of loci or nearest associated gene listed in Table 2 as demethylated at STHSC-CMP transition in the DNA of the sample from the AML subject compared to the DNA of the sample of the subject without AML indicates a negative prognosis for the subject, and wherein methylation of the minority of the plurality of loci or nearest associated gene listed in Table 2 as methylated at STHSC-CMP transition in the DNA of the sample from the AML subject compared to the DNA of the sample from the subject without AML indicates a negative prognosis for the subject.

In an embodiment, quantifying methylation is effected by recovering DNA from the blood cells digesting a first portion of the DNA with a methylation-sensitive restriction enzyme and a second portion of the DNA with a methylation-insensitive restriction enzyme, and hybridizing to a HELP microarray.

In an embodiment, quantifying methylation is effected using HpaII tiny fragment Enrichment by Ligation-mediated PCR.

In an embodiment, quantifying methylation is effected by contacting a first portion of the DNA with sodium bisulfite under conditions permitting conversion of cytosine residues of the DNA into uracils, sequencing the DNA of the first portion and of a second portion untreated with sodium bisulfite, and aligning the resultant sequences of the two portions and comparing the sequences so as to determine the extent and position of methylated nucleotides in the DNA.

In an embodiment, the methods further comprising PCR amplifying the DNA after contacting with sodium bisulfite but prior to sequencing.

In an embodiment, the plurality of loci or nearest associated gene listed in Table 2 comprises at least 5 loci or nearest associated genes. In an embodiment, the plurality of loci or nearest associated gene listed in Table 2 comprises at least 10 loci or nearest associated genes. In an embodiment, the plurality of loci or nearest associated gene listed in Table 2 comprises at least 100 loci or nearest associated genes. In an embodiment, the plurality of loci or nearest associated gene listed in Table 2 comprises at least 500 loci or nearest associated genes. In an embodiment, the plurality of loci or nearest associated gene listed in Table 2 as demethylated at STHSC-CMP transition comprises at least 5 loci or nearest associated genes. In an embodiment, the plurality of loci or nearest associated gene listed in Table 2 as demethylated at STHSC-CMP transition comprises at least 100 loci or nearest associated genes. In an embodiment, the plurality of loci or nearest associated gene listed in Table 2 as demethylated at STHSC-CMP transition comprises at least 200 loci or nearest associated genes. In an embodiment, the plurality of loci or nearest associated gene listed in Table 2 as demethylated at STHSC-CMP transition comprises at least 500 loci or nearest associated genes.

In an embodiment, the plurality of the loci or nearest associated gene listed in Table 2 as methylated at STHSC-CMP transition comprises at least 5 loci or nearest associated genes. In an embodiment, the plurality of the loci or nearest associated gene listed in Table 2 as methylated at STHSC-CMP transition comprises at least 10 loci or nearest associated genes. In an embodiment, the plurality of the loci or nearest associated gene listed in Table 2 as methylated at STHSC-CMP transition comprises at least 20 loci or nearest associated genes. In an embodiment, the plurality of the loci or nearest associated gene listed in Table 2 as methylated at STHSC-CMP transition comprises at least 30 loci or nearest associated genes. In an embodiment, the methylation is quantified as DNA cytosine methylation.

In one embodiment, the blood sample has previously been obtained from the subject. Also provided is a method as described hereinabove but further comprising the step of obtaining the blood sample from the subject.

Also provided is a method for treating a subject having acute myeloid leukemia (AML) comprising determining the prognosis of the subject, by any of the methods described herein, as positive or negative, and treating the subject with a chemotherapy if the subject has a positive prognosis or treating the subject with a non-chemotherapeutic method if the subject has a negative prognosis.

In an embodiment, the chemotherapy comprises administering an anthracycline and/or cytarabine and/or a demethylating agent, and or/a TKI. In an embodiment, the anthracycline is daunorubicin. In an embodiment, the non-chemotherapeutic method comprises an allogeneic stem cell transplantation into the subject.

In an embodiment, the non-chemotherapeutic treatment comprises all-trans-retinoic acid (ATRA), optionally with arsenic trioxide.

The practice of the present invention can employ, unless otherwise indicated, conventional techniques of molecular biology, such as PCR, e.g. see PCR: The Polymerase Chain Reaction, (Mullis et al., eds., 1994).

In some embodiments, the subject involved in methods of the invention is considered to be at risk for AML relapse. “At risk” is an art-recognized term in the medical literature. A subject who has had a remission of AML may be at risk of a relapse as determined by duration of first complete remission, adverse cytogenetics, age and FLT3 mutation status.

Further examples of isoschizomer enzyme pairs that may be used in an embodiment of the invention are the methylation sensitive and insensitive enzyme pairs listed in Table 1 of US Patent Application Publication 2010-0267021 A1, published Oct. 21, 2010, hereby incorporated by reference.

Also provided is a microarray comprising a nucleic acid probe for all, or for less than all, of the 561 loci or nearest associated genes listed in Table 2. Also provided is a kit comprising the microarray and instructions for use in determining the prognosis of an AML patient from a blood or bone marrow sample from the patient. In an embodiment, the kit further comprises reagents comprising an isoschizomer enzyme pairs having a methylation sensitive and insensitive enzyme pair.

Also provided is a kit for determining the prognosis of a subject having acute myeloid leukemia (AML), comprising
a) reagents for quantifying the methylation of DNA of a sample comprising blood cells or bone marrow cells obtained from a subject with AML at a plurality of chromosome loci, or nearest associated gene, as listed in Table 2;
b) written instructions for determining a methylation score from the methylation determined with the reagents in a);
c) written instructions for a predetermined reference amount for the same plurality of chromosome loci, or nearest associated gene, and for assigning a prognosis to the subject based on the methylation score compared to the predetermined reference amount,
wherein a methylation score at or in excess of the predetermined reference amount indicates a negative prognosis for the subject,
and wherein a methylation score below the predetermined reference amount indicates a positive prognosis for the subject.

In an embodiment of the kit, the methylation score comprises a direct or indirect measurement of the ratio of demethylated CpG residues/methylated+demethylated CpG residues of the plurality of the chromosome loci, or nearest associated gene for the DNA of a sample.

In an embodiment of the kit, the methylation is determined by a isoschizomer enzyme pair method, and wherein the kit comprises an isoschizomer enzyme pair, and wherein the methylation score is obtained by summing absolute values of the median-centered methylation values (log 2[methylation sensitive enzyme measured fragments/methylation insensitive enzyme measured fragments]) of the plurality of the chromosome loci, or nearest associated gene for the DNA of a sample.

In an embodiment of the kit, the isoschizomer enzyme pair is HpaII and MspI.

In an embodiment of the kit, the HELP assay is used to determine the methylation of the DNA.

The phrase “and/or” as used herein, with option A and/or option B for example, encompasses the individual embodiments of (i) option A, (ii) option B, and (iii) option A plus option B.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group subjectly and all possible subgroups of the main group, but also the main group absent one or more of the group members. The present invention also envisages the explicit exclusion of one or more of any of the group members in the claimed invention.

All combinations of the various elements described herein are within the scope of the invention unless otherwise indicated herein or otherwise clearly contradicted by context.

In the event that one or more of the literature and similar materials incorporated by reference herein differs from or contradicts this application, including but not limited to defined terms, term usage, described techniques, or the like, this application controls.

This invention will be better understood from the Experimental Details, which follow. However, one skilled in the art will readily appreciate that the specific methods and results discussed are merely illustrative of the invention as described more fully in the claims that follow thereafter.

### Experimental Details

### Introduction

Acute myeloid leukemia (AML) is characterized by disruption of HSC and progenitor cell differentiation. Frequently, AML is associated with mutations in genes encoding epigenetic modifiers. It was not previously known or proposed whether analysis of alterations in DNA methylation patterns during healthy HSC commitment and differentiation would yield epigenetic signatures that could be used to identify stage-specific prognostic subgroups of AML. In one embodiment a method is disclosed comprising using a nano Hpall-tiny-fragment-enrichment-by-ligation-mediated-PCR (nanoHELP) assay to compare genome-wide cytosine methylation profiles between highly purified human long-term HSC, short-term HSC, common myeloid progenitors, and megakaryocyte-erythrocyte progenitors. It was observed that the most striking epigenetic changes occurred during the commitment of short-term HSC to common myeloid progenitors, and these alterations were predominantly characterized by loss of methylation. A metric of the HSC commitment-associated methylation pattern was developed that proved to be highly prognostic of overall survival in three independent large AML patient cohorts, regardless of patient treatment and epigenetic mutations. Application of the epigenetic signature metric for AML prognosis was superior to evaluation of commitment-based gene expression signatures. Together, the data define a stem cell commitment-associated methylome that is independently prognostic of poorer overall survival in AML. The epigenetic signature is enriched for binding sites of known hematopoietic transcription factors and microRNA loci.

### Experiments

Most DNA cytosines are methylated in human HSPC—To characterize DNA cytosine methylation in early human hematopoiesis, the distribution of and changes in methylation was studied during in vivo physiologic differentiation from LTHSC, immunophenotypically characterized as Lineage (Lin)−, CD34+, CD38−, CD90+, to STHSC (Lin−, CD34+, CD38−, CD90−), to CMP (Lin−, CD34+, CD38+, CD123+, CD45RA−) to MEP (Lin−, CD34+, CD38+, CD123−, CD45RA−) (15-21). A novel method combining eight-parameter high-speed fluorescence-activated cell sorting (FACS) of primary human bone marrow cells with an optimized Hpall-tiny-fragment-Enrichment-by-Ligation-mediated PCR (nano-HELP) assay (22-26) was used. This approach permitted examination of single individuals' stem cells isolated as biological replicates, i.e. without pooling of samples prior to analysis. It was possible to analyze DNA cytosine methylation in rare, highly purified human HSPC populations. Globally, it was found that the majority of DNA cytosines in human LTHSC, STHSC, CMP, and MEP (76%-81% of loci) from healthy individuals were methylated. Methylation was quantitatively compared across all loci between the stages of differentiation. Interestingly, it was found that there was a highly significant reduction in median DNA cytosine methylation specifically at the stem cell to progenitor (STHSC to CMP) transition (p<2.2×10−16, Mann-Whitney test).

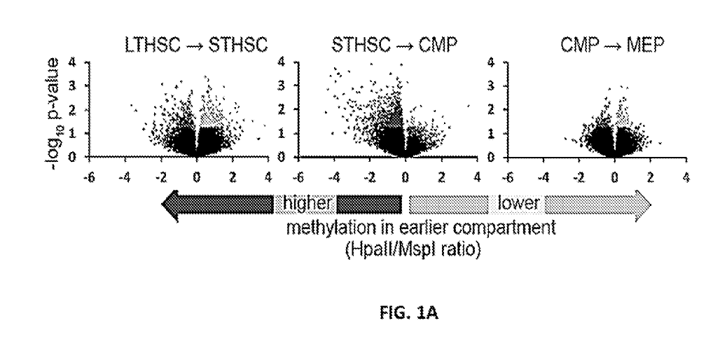

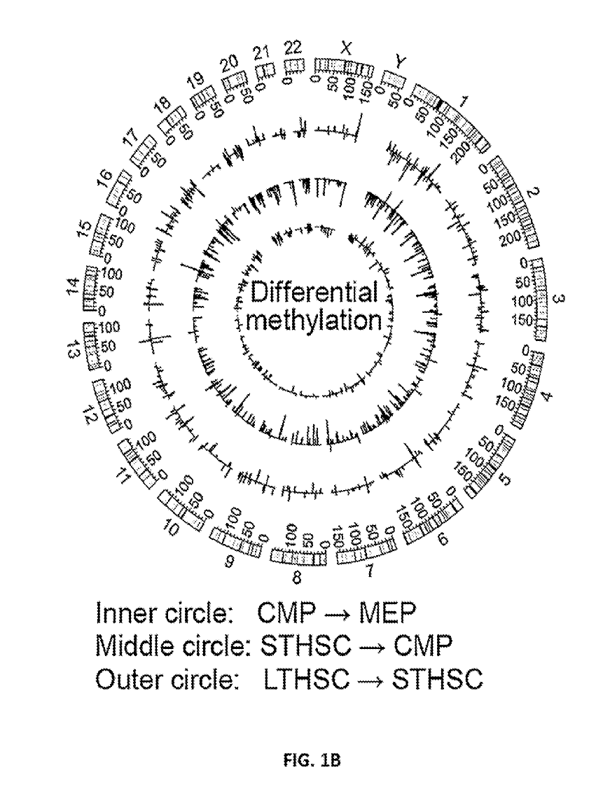

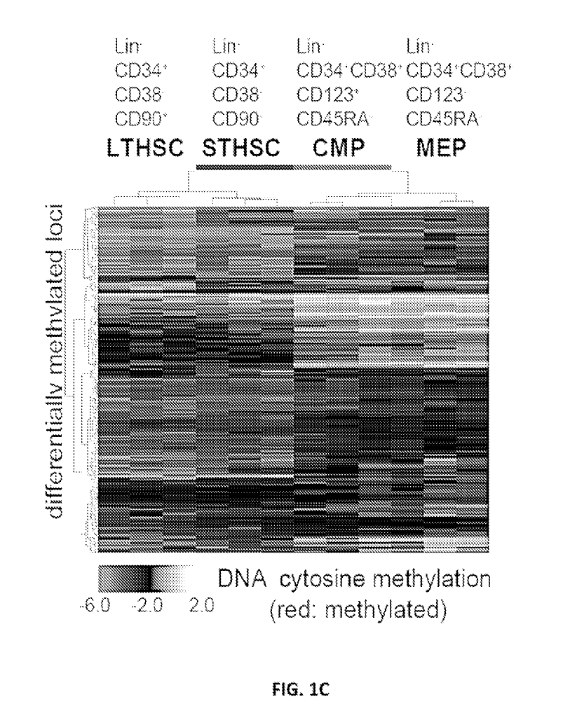

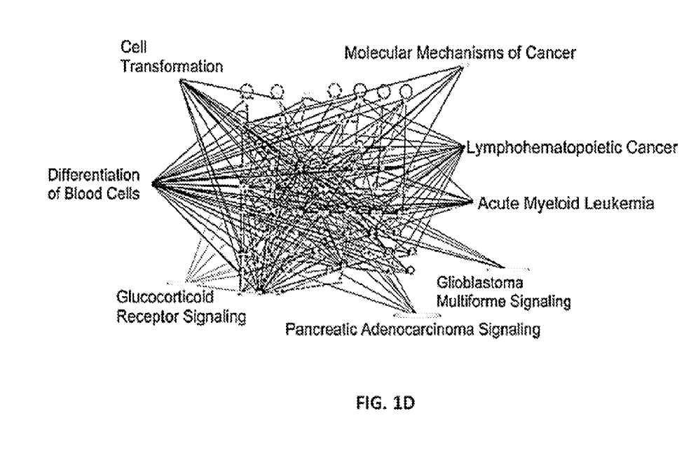

Dynamic changes in DNA cytosine methylation during HSC commitment—To characterize the dynamics of cytosine methylation during HSC commitment, changes in the methylation status at the level of individual loci were investigated and methylation in LTHSC was compared to methylation in STHSC, STHSC to CMP, and CMP to MEP. The comparison between LTHSC and STHSC showed 509 significantly differentially methylated loci (p<0.05). Demethylation was observed in 40% (205/509) of these loci during transition from the LTHSC to the STHSC compartment, whereas 60% (304/509) were more methylated in STHSC compared to LTHSC. At the transition from STHSC to CMP, the step of definitive commitment of HSC, a total of 793 differentially methylated loci were observed. However, in stark contrast to the nearly balanced hypo- and hypermethylation of loci between LTHSC and STHSC, 95% (757/793) of differentially methylated loci in STHSC were more methylated than in CMP, whereas only 5% (36/793) were less methylated. The transition from CMP to MEP was accompanied by balanced hypo- and hypermethylation with 127 (52%) loci showing higher and 116 (48%) loci showing lower methylation in the CMP compartment (FIG. 1A). Changes occur without apparent focus throughout the genome (FIG. 1B). The findings show that in human HSC demethylation particularly occurs at the commitment step from STHSC to CMP. This had not been described thus far.

A stem cell commitment-associated epigenetic signature distinguishes human HSC and progenitor cell subsets—To identify loci with most significant methylation changes across the assessed differentiation stages, significance analysis for microarrays (SAM) was performed on loci that showed differentiation-specific methylation changes independent of the variation between biological replicates, in analogy to a recently published approach (7). This resulted in a set of 561 loci that distinguished between the four investigated stages of human HSPC development (FIG. 1C). The most prominent distinction was observed at the transition from stem cells (STHSC) to progenitor cells (CMP), consistent with the analysis of changes in DNA cytosine methylation during stem cell commitment described in FIG. 1A. The signature mainly consisted of loci that were significantly demethylated during commitment from STHSC to CMP (516/561 loci, 92.0%). Interestingly, this stem cell commitment-associated epigenetic signature was enriched in loci associated with several genes that are commonly implicated not only in human HSC function and commitment but also in leukemogenesis, such as CEBPA (27-29), E2F1 (30), KRAS (31, 32), WEE1 (33), as well as a non-coding transcript, MIRLET7B (34) (Table 2). Given emerging evidence that microRNAs play an essential role in both normal hematopoiesis and leukemogenesis (35-38) additional microRNA transcripts were assessed in the vicinity of the methylation probes on the array. Using ‘miRBase’ it was found that a number of microRNAs that were associated with significant epigenetic changes (Table 2). Ingenuity pathway analysis using the significant constituents of this methylation signature revealed an enrichment of genes involved in the function and disease characteristics “Differentiation of Blood Cells” (p=8.39E-43), “Lymphohematopoietic Cancer” (p=3.2E-12), specifically “AML” (p=2.47E-06), and “Cell Transformation” (p=1.41E-12). The top five canonical pathways were “AML Signaling”, “Molecular Mechanisms of Cancer”, “Glioblastoma Multiforme Signaling”, “Pancreatic Adenocarcinoma Signaling”, and “Glucocorticoid Receptor Signaling” (FIG. 1D). Taken together, significant changes in DNA cytosine methylation during human HSC commitment occur at genomic loci involved in hematopoietic differentiation and in hematological malignancies.

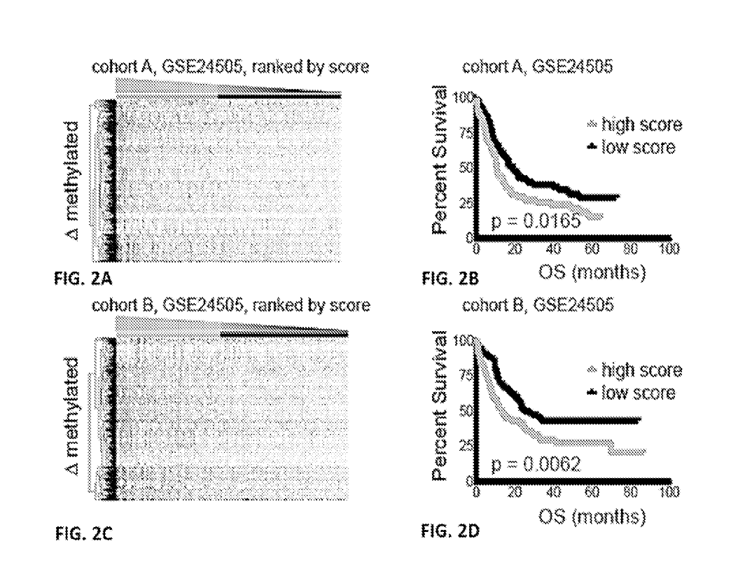

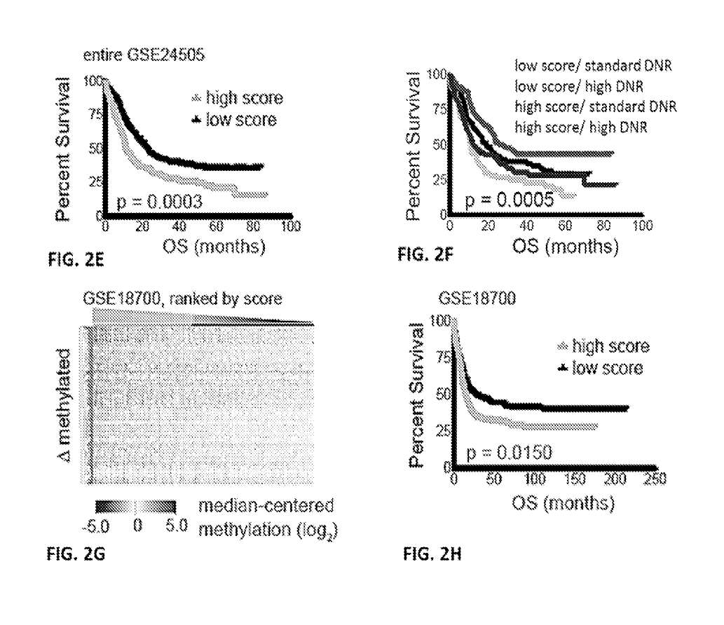

Stem cell commitment-associated epigenetic signature is prognostic for overall survival in AML—Pathway analysis of the epigenetic signature showed an enrichment of genes implicated in systemic disorders of hematopoietic development. It was sought to determine whether the methylation status of this set of 561 stem cell commitment-associated loci derived from healthy human HSPC was affected in AML, a disease associated with epigenetic deregulation in HSPC (1). A signature score was developed based on the methylation of the 561 loci defined by the stem cell commitment-associated epigenetic signature. Additionally, data from clinical trials of patients with AML were analyzed. Three published independent cohorts of patients were identified for which DNA methylation data, gene expression data, as well as data on overall survival (OS) and mutational characteristics were available (4, 39-42). To assess the prognostic impact of this epigenetic signature was developed, we associated OS of patients with their score. This approach was tested on one cohort from a prospective randomized clinical trial that compared two different doses of daunorubicin (41). In the cohort receiving the standard, lower dose daunorubicin, a low stem cell commitment-associated epigenetic signature score was associated with increased OS (HR=1.537, 95% CI=1.086-2.245, p=0.0165, log-rank test, FIG. 2A, B). Patients in the group with lower epigenetic signature scores showed a median OS of 19.0 months, compared to 10.8 months in the group with higher epigenetic scores. Next, the stem cell commitment-associated epigenetic signature score was applied to the group of patients that received a higher dose of daunorubicin (41). The association of the stem cell commitment-associated epigenetic signature score with OS was also observed in this cohort of patients (HR=1.691, 95% CI=1.169-2.552, p=0.0062, FIGS. 2C, D). Median OS in the group with low epigenetic signature score was 25.4 months, compared to 13.2 months in the high scoring group. Of note, the significant association of high epigenetic signature score with poor outcome persisted upon combination of the two treatment arms of this trial (FIG. 2E, median OS 11.1 months for patients with a high versus 22.8 months for patients with a low score, HR=1.609, 95% CI=1.258-2.143, p=0.0003).

To independently assess the association of the loci from the stem cell commitment-associated epigenetic signature with clinical outcome, Globaltest analysis was performed (43), using these loci as covariates. This confirmed the significant association of the 561-loci-classifier with OS (p=0.000697). In a multivariate Cox proportional hazard regression analysis (44) which included the epigenetic score in addition to the well-established factors cytogenetic and molecular risk stratification (3) and age, the epigenetic score remained independently and significantly associated with OS (Table 1). As depicted in the overlay of the survival curves from FIGS. 2B and D, patients with a low epigenetic signature score receiving high levels of daunorubicin had a significantly better OS than patients from the other groups (FIG. 2F, p=0.0005), whereas patients in the three remaining groups did not show a statistically significant difference in their respective OS.

Additionally, the power of the epigenetic score in a third, independent cohort of patients with AML was validated. For this, published clinical and methylation data from patients from four clinical trials included in a study from the Dutch-Belgian Cooperative Trial Group for Hematology Oncology (HOVON) group (4, 39, 40) were analyzed. In this study, patients with a low epigenetic score again had a significantly better OS than those with a high score (median survival 28.1 months versus 14.9 months; HR=1.390, 95% CI=1.069-1.838, p=0.0150) (FIGS. 2G and H). Globaltest analysis (43) of this cohort independently confirmed significant association of the signature score with survival (p=0.000335).

Taken together, the methylation status of the commitment-associated loci identified in human HSPC from healthy individuals showed independent prognostic power in human AML in a total of 688 patients.

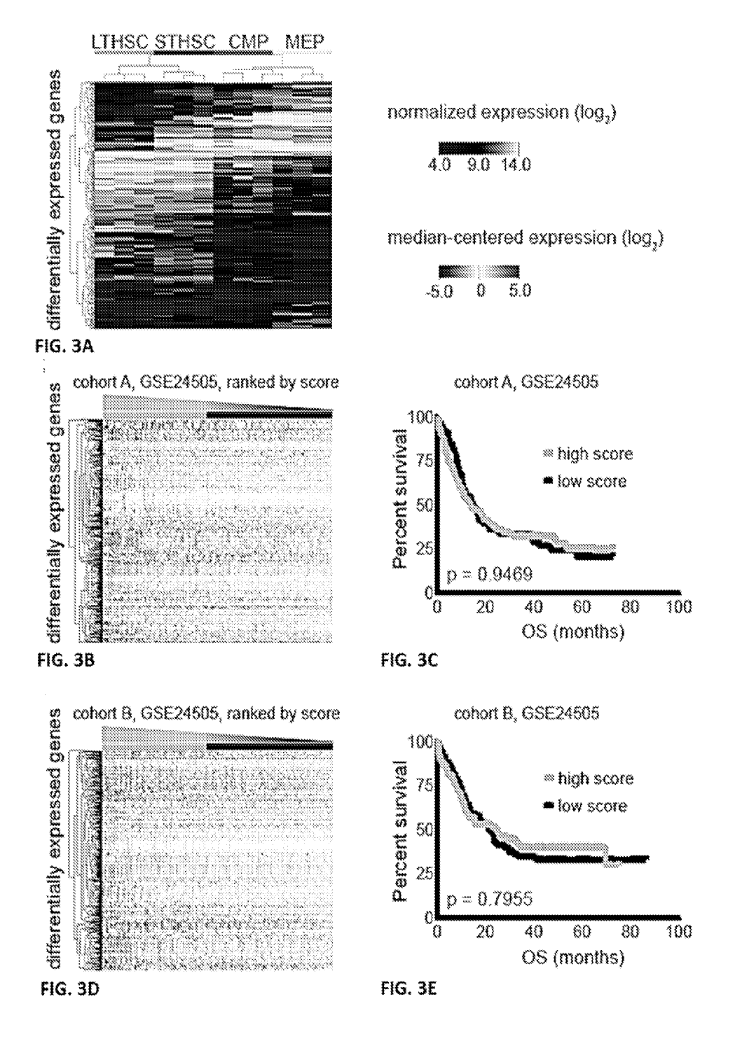

Low correlation of commitment-associated gene expression signature with AML patient outcome—Previous studies have defined gene expression signatures predictive of OS of patients with AML (45-47). Therefore, it was sought to determine whether a gene expression signature constructed in analogy to the epigenetic signature had comparable prognostic potential in the AML cohorts studied. It was first determined whether differentiation-specific gene expression changes were independent of the variation between biological replicates by SAM. Expression of the identified transcripts distinguished between the four investigated stages of human HSPC development (FIG. 3A). The approach chosen to associate the epigenetic signature with OS was repeated and applied to this gene expression signature to the AML patient cohorts. The signature consisted of 530 genes that were differentially expressed in the analyzed stem and progenitor cells from healthy human individuals (FIG. 3B, D). No significant correlation of the stem cell commitment-associated gene expression signature with OS was observed in either AML treatment group (FIG. 3C, E). Association of gene expression signatures with outcome using globaltest as an alternative algorithm revealed a significant association of these genes with OS only in the combined Eastern Cooperative Oncology Group (ECOG) cohort (p=0.00168) but not in the HOVON cohort (p=0.363). While a published HSC gene expression signature (46) was associated with OS in the ECOG cohort (p=0.00202, globaltest), the association of a leukemia stem cell gene expression signature (46) with OS missed significance (p=0.0821, globaltest) as did an additional leukemic stem cell gene expression signature (45) (p=0.257, globaltest). These findings suggest that the stem cell commitment-associated epigenetic signature is a more robust indicator of OS than a stem cell-commitment-associated gene expression signature obtained in an identical, unbiased fashion.

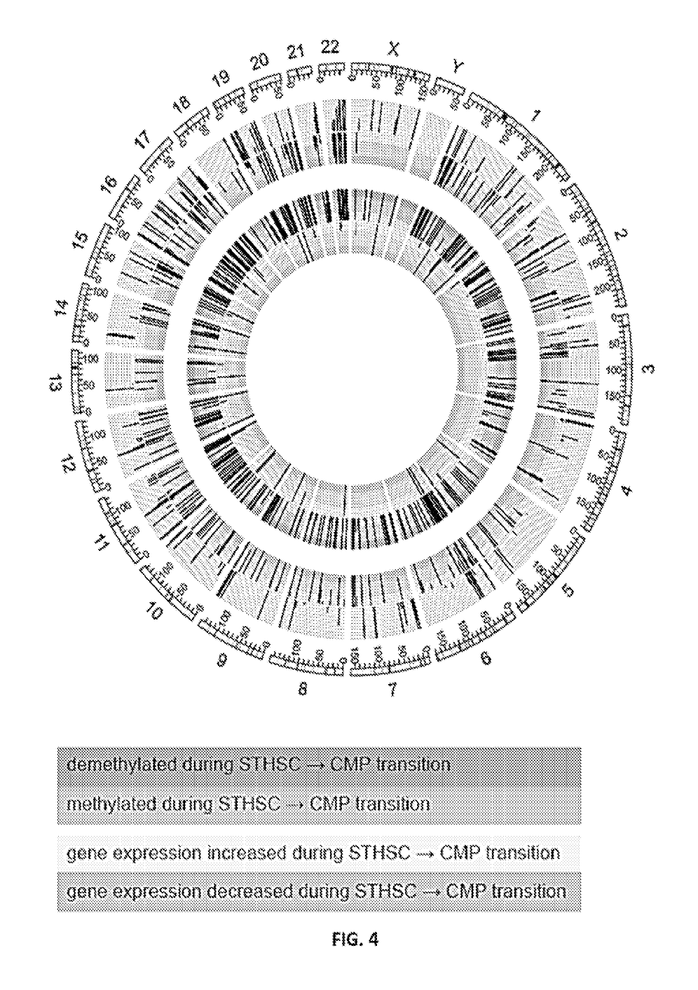

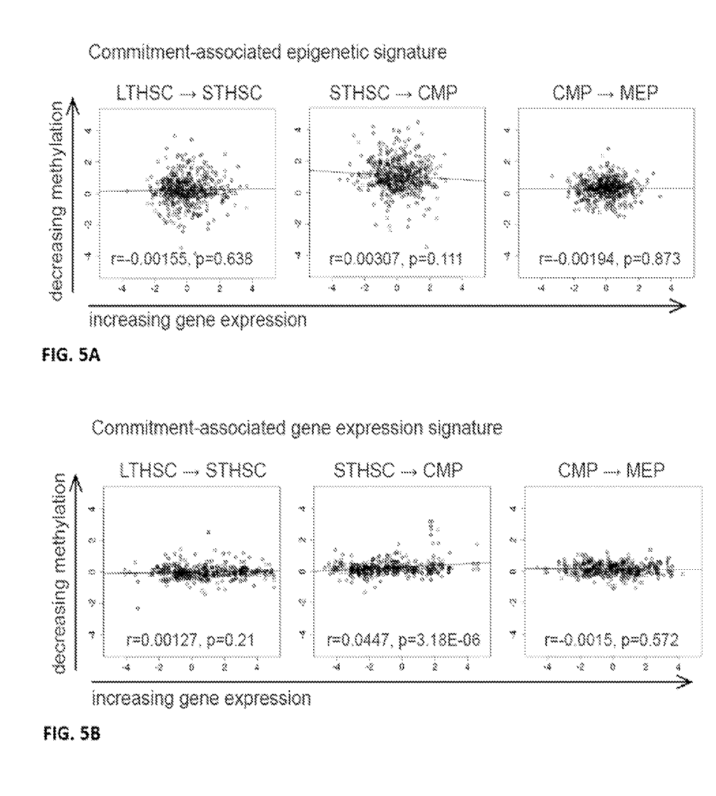

Correlation of methylation and gene expression changes between stages of human hematopoietic stem cell commitment—DNA cytosine methylation has been associated with regulation of transcription. Promoters of developmental genes, as well as promoters of housekeeping genes can be silenced by hypermethylation (48) while gene bodies have been reported to be methylated following increased transcription of the respective gene (49). Methylation and gene expression were correlated during the respective HSPC transitions. Besides locus-specific inverse correlation between decreasing methylation and increasing gene expression (FIG. 4, FIG. 5A upper right quadrant), increasing methylation and decreasing gene expression (FIG. 5A, lower left quadrant) loci were found with a positive correlation between decreasing methylation and decreasing gene expression (FIG. 5A, upper left quadrant), and increasing methylation and increasing gene expression (FIG. 5A, lower right quadrant). Conversely, a significant correlation between decrease of cytosine methylation and increase in gene expression at the STHSC to CMP transition appeared when correlating the commitment-associated gene expression signature with nearby CpG loci (FIG. 5B). Changes in methylation at an earlier transition did not significantly associate with changes in gene expression at a later transition (e.g. methylation during transition from LTHSC to STHSC compared to gene expression during transition from STHSC to CMP, data not shown). Taken together, the epigenetic signature is not universally correlated with gene expression, although there are certain loci that show correlation or inverse correlation. Yet, at the STHSC to CMP transition an inverse correlation between gene expression and associated methylation changes can be observed. Changes in expression of the genes associated with the epigenetic stem cell-commitment associated signature were not prognostic for outcome in AML patients (p=0.133, ECOG cohort, Globaltest). Of note, mutations of genes known to directly affect DNA methylation, such as IDH1, IDH2, TET2, and DNMT3A, were not enriched in either the high or low scoring group. Finally, it was investigated whether specific DNA motifs were enriched around the constituents of the epigenetic signature, which could provide mechanistic insights into the regulation of these loci. Using HOMER transcription binding site analysis (50), a significant enrichment was observed of consensus binding sites for several essential transcription factors involved in hematopoietic differentiation (most notably GATA transcription factors, Maf family members, KLF4, and Smad2 (51-53)) in the epigenetic signature.

### Discussion

Perturbed epigenetic regulation of differentiation from HSC to mature blood cells can result in a block in cellular differentiation, clinically apparent in hematopoietic malignancies such as AML (1). To study epigenetic regulation during earliest human hematopoiesis, the status of and changes in DNA cytosine methylation during in vivo differentiation of human HSC was analyzed. To this end, a novel technique was developed that enabled characterization of DNA cytosine methylation from prospectively isolated highly enriched human HSC from single individuals in small numbers. Prospective isolation of human HSPC was coupled with a modified HELP assay, the so-called nano-HELP (22-26). It was found that most DNA cytosines in human LTHSC, STHSC, CMP, and MEP are methylated, in agreement with findings in other vertebrate somatic stem cells and differentiated tissues (5-7, 54). The findings show that, while mean methylation levels are comparable to those found in murine HSC (7), in human HSC demethylation particularly occurs at the commitment step from STHSC to CMP (FIG. 1A). This has not been described thus far. Furthermore, our data define specific loci with dynamic changes in methylation during human HSPC differentiation. These loci represent a stem cell commitment-associated epigenetic signature that clusters the subsequent stages of HSC differentiation (FIG. 1C), and is enriched in genes associated with hematopoietic development and also leukemogenesis, particularly AML (FIG. 1D). Therefore, it was assessed whether the methylation status at these loci would have clinical implications in human AML. Indeed, it was found that this signature was able to classify three independent cohorts of patients with AML from prospective clinical trials into groups with superior or significantly inferior OS. Patients treated with standard chemotherapy with a low stem cell commitment-associated epigenetic signature score reached significantly longer OS than patients with a high score. The power of this score was assessed using data from a second cohort of AML patients treated with an experimental approach (41) and an even stronger distinction was found between the groups (FIG. 2). This is in contrast to some currently used mutational markers (3), and suggests a high degree of robustness of the prognostic value of the stem cell commitment-associated epigenetic signature. Multivariate analysis demonstrated an independent association of the epigenetic score with OS, and no enrichment of mutations of known modifiers of DNA methylation was detected in either the high or low scoring group. The overlay of survival curves from the different clinical cohorts (FIG. 2F) suggests that the epigenetic signature might serve as a predictor for OS particularly in AML patients receiving higher doses of daunorubicin. A third independent cohort of patients with AML studied by the HOVON group (39, 40) also segregated into better and worse prognosis on the basis of the epigenetic stem cell commitment-associated score, further demonstrating the robustness and prognostic potential of this score. Taken together, the epigenetic stem cell commitment signature was validated in three independent cohorts of AML patients, with a total of 688 patients. Of note, in each of these cohorts median survival was approximately doubled in patients with low signature score, even in the cohort that was treated with higher dose daunorubicin, indicating the robustness of the prognostic value of this signature. Similarly derived gene expression signatures were not able to achieve the robustness that was observed using the epigenetic signature.

Recent studies have linked changes in methylation to the regulation of microRNAs, and one microRNA transcript, MIRLET7, was identified in the signature; in addition, several other microRNA genes were located adjacent to the differentially methylated region (DMR).

Sequence analysis of the DMR regions revealed a significant enrichment of motifs for transcription factors that were previously shown to be implicated in hematopoietic differentiation and leukemogenesis, such as GATA factors, MAFF and KLF4. For instance, it was recently shown that erythroid differentiation is accompanied by functional demethylation of essential erythropoietic genes, including GATA1 (6, 55). In addition, maintenance of hematopoietic stem cell programs and prevention of activation of differentiation programs are controlled by DNA methylation (8).

Analyses were performed on DNA from highly enriched HSPC, thus avoiding the measurement of DNA cytosine methylation and gene expression from heterogeneous cell populations. In addition, analyzing cells from single donors, as opposed to pooling cells from multiple donors, permitted derivation of changes propagated through various differentiation stages in individuals, in addition to changes that occurred in a stage-specific manner across all individuals studied. Furthermore, an exhaustive high quality dataset that included both data on DNA cytosine methylation in leukemic blasts and clinical data including a detailed description of risk groups and overall survival from a prospective randomized clinical trial were accessed (41). These data have been the basis for numerous analyses (3, 42, 56). The HELP assay has a bias towards CpG-rich sites, in effect concentrating on promoter regions. The performance of the HELP assay in CpG-poor regions is reduced compared to bisulfite conversion based methods.

In summary, the findings presented here identify a large fraction of CpG dinucleotides in human HSC as methylated, show a human-specific methylation decrease specifically during STHSC to CMP commitment, and reveal a stem cell commitment-associated epigenetic signature as robustly and independently prognostically significant for OS of AML patients.

### Methods and Materials

Bone marrow samples: Bone marrow samples from healthy individuals were obtained from AllCells LLC.

High-speed multi-parameter fluorescence-activated cell sorting (FACS): FACS of human HSPC populations was performed as described before (15-17, 19-21, 25). Mononuclear cells from bone marrow aspirates were isolated by density gradient centrifugation. CD34+ cells were enriched by immunomagnetic beads (Miltenyi Biotech). The resulting cells were lineage depleted (Lin−) using PE-Cy5 (Tricolor)-conjugated monoclonal antibodies against CD2, CD3, CD4, CD7, CD10, CD11b, CD14, CD15, CD19, CD20, CD56, and Glycophorin A (all BD Biosciences). Further distinction into HSPC subsets was performed using fluorochrome-conjugated antibodies against CD34, CD38, CD90, CD45RA, and CD123 (all eBioscience). LTHSC (Lin−, CD34+, CD38−, CD90+), STHSC (Lin−, CD34+, CD38−, CD90−), CMP (Lin−, CD34+, CD38+, CD123+, CD45RA−), and MEP (Lin−, CD34+, CD38+, CD123−, CD45RA−) were sorted into RLT extraction buffer (Qiagen). Flow cytometric analysis and cell separation were performed on a FACSAriaII special order system (Becton Dickinson) equipped with 4 lasers (407 nm, 488 nm, 561/568 nm, 633/647 nm).

Preparation of nucleic acids: After sorting into RLT buffer (Qiagen), homogenization of the cells was achieved by passing the cells five times through a needle. Simultaneous harvest of RNA and genomic DNA was achieved with the AllPrep kit (Qiagen) following the instructions of the manufacturer. Total RNA was linearly amplified and transcribed with the MessageAmp Kit AM1751 (Ambion/Life Technologies) prior to microarray gene expression analysis following the NimbleGen Arrays User's Guide (NimbleGen). Integrity of RNA and cDNA was verified at each step of amplification using the Agilent Bioanalyzer 2100 (Agilent).

DNA methylation analysis by nano-HELP: Methylation analysis by the HELP assay (22, 57-59) and a modified protocol to successfully work with low genomic DNA yield from low numbers of sorted stem and progenitor cells have been described (24, 25). Integrity of genomic DNA of high molecular weight was assured by electrophoresis for all samples used. HpaII or MspI (NEB) digestions of genomic DNA were performed overnight prior to overnight ligation of the HpaII adapter with T4 ligase. PCR amplified adapter-ligated HpaII or MspI fragments were submitted to Roche-NimbleGen. Labeling and DNA hybridization onto a human hg17 custom designed oligonucleotide array (50mers) was carried out. The 2005-07-20_HG17_HELP_Promoter array covers 25,626 HpaII amplifiable fragments (HAF) at gene promoters, defined as regions 2 kb upstream and downstream of transcriptional start sites (TSS). EpiTyper by MassArray (Sequenom) was used to confirm methylation of selected loci as described (23, 60).

Microarray quality control: Uniformity of hybridization was evaluated by adapting a published algorithm (61) for the NimbleGen platform. Hybridizations with strong regional artifacts were discarded and repeated. Normalized signal intensities from each array were compared with a 20% trimmed mean of signal intensities across all arrays in that experiment. Arrays with significant intensity bias that could not be explained by the biology of the sample were excluded.

HELP data processing: Signal intensities at each HAF were calculated as 25% trimmed mean of their component probe-level signal intensities. Any fragments found within the level of background MspI signal intensity (equaling 2.5 mean-absolute-difference, MAD) above the median of random probe signals were regarded “failed”. These “failed” loci represent the population of fragments that did not amplify by PCR. Loci were designated “methylated” when the level of HpaII signal intensity was indistinguishable from background as described for MspI. Fragments successfully amplified by PCR, i.e. distinguishable above background, were subjected to normalization. For this, an intra-array quantile approach was used: HpaII/MspI ratios are aligned across density-dependent sliding windows of fragment size-sorted data. The log 2(HpaII/MspI) was used as a representative for methylation and analyzed as a continuous variable. If the centered log 2(HpaII/MspI) ratio was <0, the corresponding fragment was considered methylated. It was considered hypomethylated in cases where log 2(HpaII/MspI) was >0.

Gene expression profiling: Gene expression profiling was performed on NimbleGen HG18 arrays (design name 2006-08-03_HG18_60mer_expr, Roche-NimbleGen). Profiling was performed by the Epigenomics Shared Facility, Albert Einstein College of Medicine.

Meta-analysis of the GSE24505 AML data set: Previously published data for gene expression (Nimblegen 2005-04-20_Human_60mer_lin2 arrays), and DNA methylation (2005-07-20_HG17_HELP_Promoter arrays) were retrieved from the GEO server (http://www.ncbi.nlm.nih.gov/geo/query/acc.cgi?acc=GSE24505). Additional annotations were extracted from these files. The methylation status of respective loci could be directly compared between the data describing human HSPC that we analyzed and the published GSE24505 AML data due to identical platforms.

Statistical analysis: HELP loci were annotated using UCSC annotations for hg17. Means of locus-specific methylation between consecutive HSPC subsets were compared using Student's two-sided t-test for unpaired samples. Significance was assumed when p<0.05. Significance analysis for microarrays (SAM) was performed using Multiple Experiment Viewer as was supervised clustering using Euclidean distance correlation with complete linkage. SAM (q<0.015) was performed on the values of the 4 cell populations that remained significant after an initial SAM had filtered probes in which the difference between replicates was more significant than the difference between stages of differentiation. A similar approach to account for variability in analyses of DNA cytosine methylation has recently been published (7). Survival data and corresponding methylation values have previously been published (41, 42). An epigenetic score was calculated by summing absolute values of the median-centered methylation values (log 2[HpaII/MspI]) of the 561 signature loci for each patient sample. Samples from ECOG (GSE24505) and HOVON (GSE18700) studies (4, 42) were ranked and uniformly dichotomized according to the 55th percentile into patients with a low and those with a high signature score. An association of this score with Kaplan-Meier-survival estimates (62) was probed by the log rank test and assumed to be statistically significant when p<0.05. The association of individual methylation loci and genes in this set of patient samples was probed by globaltest (43) after linear transformation to obtain positive values, similarly to a recently published analysis (63). Gene expression analysis was performed in an identical fashion, with q<0.2. Ingenuity (Ingenuity Systems) was used for pathway analysis. After Benjamini-Hochberg-correction, Top Bio Functions that were significantly (p<0.05) associated with the 561 constituents of the epigenetic commitment-associated stem cell signature were entered into a pathway generator. The top five canonical pathways and the top three characteristics in function and disease were chosen for display. Circos plots were prepared following the instruction at http://circos.ca. To perform correlation analyses between methylation probes and gene expression changes, as well as globaltest analyses of gene expression signatures from various microarray platforms, all probes were remapped to hg19 using liftOver (genome.ucsc.edu/cgi-bin/hgLiftOver), and remapped probes were associated with overlapping hg19 RefSeq genes (retrieved from UCSC table browser genome.ucsc.edu/cgi-bin/hgTables, refGene table, retrieved Sep. 18, 2012)) using bedtools intersect, and closest non-overlapping genes were associated using bedtools closest. Additional identifiers of these genes were retrieved from ENSEMBL BioMart using biomaRt in R/Bioconductor to match probe identifiers across various microarray platforms (Nimblegen HG18 for the ECOG data set (42), Nimblegen HG17 for healthy human HSPC, Affymetrix U133plus2.0 for the signatures published by Eppert et al. (46), Entrez IDs for those published by Gentles et al. (45)). Collapsing of multiple probes, where necessary, was performed using the collapseRows function in the R/Bioconductor WGCNA package. Genomic coordinates of pre-microRNA in the hg19 genome were retrieved from miRBase (mirbase.org/pub/mirbase/20/genomes/hsa.gff2), miRBase v20, date: May 24, 2013, genome build: GRCh37.p5, NCBI_Assembly:GCA_000001405.6).

Data were compared by 2-sided t test for unpaired samples, or by significance analysis for microarrays (SAM) using Multiple Experiment Viewer (version 4.8) and q-value thresholds as indicated. To determine the association of DNA methylation or RNA expression signatures with overall survival, Kaplan-Meier survival analysis was performed and survival differences between groups were assessed with the log-rank test. Alternatively, globaltest analysis was performed. Univariate and multivariate analyses of hazard ratios were performed using the Cox proportional hazards model. Survival analyses were performed with R/Bioconductor software and the packages globaltest, survival, eha, and MASS, or with GraphPad Prism software (version 6). P-values<0.05 were considered significant.

TABLE 1
Multivariate analysis using the epigenetic stem cell commitment signature
score, cytogenetic and molecular risk stratification, age, and treatment
of the patients from GSE24505 as covariates. Score, risk and treatment
were considered as categorical values. Survival analysis was performed
using a Cox proportional hazards model.

| | HR (95% CI), p-value | |
|---|---|---|
| epigenetic score | 0.6856 (0.5247-0.8957), 0.0056 | GSE24505 |
| (high = 1) | | |
| intermediate risk | 2.0328 (1.3415-3.0803), 0.0008 | |
| unfavorable risk | 4.2794 (2.8662-6.3893), 1.17e−12 | |
| age (>=46 = 1) | 0.6905 (0.5267-0.9052), 0.0073 | |
| treatment arm (A = 1) | 0.7682 (0.5889-1.0021), 0.0518 | |

TABLE 2

| | | | | nearest | demethylated at | methylated at | associated |
|---|---|---|---|---|---|---|---|
| | | | | associated | STHSC-CMP | STHSC-CMP | mirBase |
| ID | chromosome | start | end | gene | transition | transition | miRNA |
| NR_027693 | chr1 | 957906 | 958178 | C1orf170 | • | | |
| NM_003327 | chr1 | 1189309 | 1189551 | TNFRSF4 | • | | |
| NM_003327 | chr1 | 1189552 | 1190026 | TNFRSF4 | • | | |
| NM_058167 | chr1 | 1237557 | 1237908 | UBE2J2 | • | | |
| NM_058167 | chr1 | 1249687 | 1250185 | UBE2J2 | • | | |
| NM_024848 | chr1 | 2354082 | 2354504 | MORN1 | • | | |
| NR_033711 | chr1 | 3687181 | 3688411 | TP73-AS1 | • | | |
| NM_024654 | chr1 | 6549100 | 6549357 | NOL9 | • | | |
| NM_015164 | chr1 | 15754221 | 15755055 | PLEKHM2 | • | | |
| NM_014424 | chr1 | 16090875 | 16091622 | HSPB7 | • | | |
| NR_027504 | chr1 | 16716927 | 16717382 | MST1P2 | • | | |
| NM_003689 | chr1 | 19382942 | 19383319 | AKR7A2 | • | | |
| NM_032236 | chr1 | 21855465 | 21856098 | USP48 | • | | |
| NM_005529 | chr1 | 22010022 | 22010782 | HSPG2 | • | | |
| NM_030634 | chr1 | 23440314 | 23440761 | ZNF436 | • | | |
| NM_004091 | chr1 | 23603359 | 23604042 | E2F2 | • | | |
| NM_003680 | chr1 | 32953197 | 32954032 | YARS | • | | |
| NM_003680 | chr1 | 32954033 | 32954680 | YARS | • | | |
| NM_145047 | chr1 | 36585404 | 36586033 | OSCP1 | • | | |
| NR_024270 | chr1 | 45438795 | 45439054 | LOC400752 | • | | |
| NR_038953 | chr1 | 53416058 | 53416293 | LOC100507564 | | • | |
| NM_016126 | chr1 | 54122232 | 54122724 | HSPB11 | • | | |
| NR_026782 | chr1 | 54817457 | 54819004 | HEATR8 | • | | |
| NM_001902 | chr1 | 70589060 | 70590030 | CTH | • | | |
| NM_198549 | chr1 | 77956930 | 77957172 | FAM73A | • | | |
| NM_006536 | chr1 | 86599385 | 86600099 | CLCA2 | • | | |
| NR_026988 | chr1 | 87307708 | 87308405 | LOC339524 | • | | |
| NR_026988 | chr1 | 87308406 | 87308954 | LOC339524 | • | | |
| NR_026987 | chr1 | 87308955 | 87309663 | LOC339524 | • | | |
| NM_144988 | chr1 | 95250505 | 95251771 | ALG14 | | • | |
| NM_001144822 | chr1 | 116826399 | 116827849 | CD58 | • | | |
| NM_005105 | chr1 | 142995970 | 142996399 | RBM8A | • | | |
| NM_178348 | chr1 | 149612211 | 149613164 | LCE1A | | • | |
| NM_006556 | chr1 | 151723132 | 151723767 | PMVK | • | | |
| NM_001018016 | chr1 | 151975007 | 151975698 | MUC1 | • | | |
| NM_001162384 | chr1 | 152761420 | 152761690 | ARHGEF2 | • | | |
| NM_024540 | chr1 | 153527535 | 153527805 | MRPL24 | • | | |
| NM_004833 | chr1 | 155859524 | 155860191 | AIM2 | | • | |
| NM_020335 | chr1 | 157197685 | 157197955 | VANGL2 | • | | |
| NM_005099 | chr1 | 157980315 | 157980942 | ADAMTS4 | • | | |
| NM_053053 | chr1 | 163577801 | 163578340 | TADA1 | • | | |
| NR_037845 | chr1 | 170176093 | 170177170 | LOC100506023 | • | | |
| NM_198493 | chr1 | 170370693 | 170372081 | ANKRD45 | • | | |
| NM_001200050 | chr1 | 179490846 | 179491482 | NPL | • | | |
| NM_007212 | chr1 | 181744853 | 181745145 | RNF2 | • | | |
| NM_007212 | chr1 | 181745146 | 181745897 | RNF2 | • | | |
| NM_014388 | chr1 | 206388104 | 206389080 | DIEXF | • | | |
| NM_001164688 | chr1 | 208053353 | 208053680 | RD3 | • | | |
| NM_001164688 | chr1 | 208053681 | 208054061 | RD3 | • | | |
| NR_024485 | chr1 | 224224936 | 224225388 | LOC100130093 | • | | |
| NM_145257 | chr1 | 225785590 | 225785825 | C1orf96 | • | | |
| NM_004481 | chr1 | 226508139 | 226509317 | GALNT2 | • | | |
| NM_004485 | chr1 | 232140050 | 232141799 | GNG4 | • | | |
| NM_019891 | chr1 | 232771598 | 232772990 | ERO1LB | | • | |
| NR_026833 | chr2 | 6072507 | 6073401 | LOC400940 | | • | |
| NM_003597 | chr2 | 10097327 | 10097750 | KLF11 | • | | |
| NM_002539 | chr2 | 10539882 | 10540677 | ODC1 | • | | |
| NM_001006946 | chr2 | 20347390 | 20348252 | SDC1 | • | | |
| NM_175629 | chr2 | 25506723 | 25507149 | DNMT3A | • | | |
| NM_004802 | chr2 | 26590216 | 26591090 | OTOF | • | | |
| NM_001168364 | chr2 | 27575821 | 27576237 | KRTCAP3 | • | | |
| NM_001168364 | chr2 | 27576238 | 27576601 | KRTCAP3 | • | | |
| NM_001199139 | chr2 | 32402061 | 32402439 | NLRC4 | • | | |
| NM_144736 | chr2 | 37370960 | 37371797 | C2orf56 | • | | |
| NM_001001521 | chr2 | 63979435 | 63979829 | UGP2 | • | | |
| NM_181784 | chr2 | 65571224 | 65571496 | SPRED2 | • | | |
| NM_181784 | chr2 | 65575553 | 65576585 | SPRED2 | • | | |
| NM_001190265 | chr2 | 68201971 | 68202668 | C1D | • | | |
| NM_001111101 | chr2 | 68458347 | 68458608 | CNRIP1 | • | | |
| NM_017880 | chr2 | 70329727 | 70330679 | C2orf42 | | • | |
| NM_001206840 | chr2 | 85467468 | 85467949 | TGOLN2 | • | | |
| NM_017750 | chr2 | 85492695 | 85492916 | RETSAT | • | | |
| NM_012483 | chr2 | 85831349 | 85832243 | GNLY | • | | |
| NM_001079 | chr2 | 97787264 | 97787854 | ZAP70 | • | | |
| NM_014044 | chr2 | 98684341 | 98684820 | UNC50 | • | | |
| NM_032411 | chr2 | 106139577 | 106140059 | C2orf40 | • | | |
| NM_006343 | chr2 | 112370475 | 112371817 | MERTK | • | | |
| NM_173843 | chr2 | 113590796 | 113591607 | IL1RN | • | | |
| NM_001105198 | chr2 | 120151906 | 120152205 | TMEM177 | • | | |
| NM_005291 | chr2 | 128119819 | 128120032 | GPR17 | • | | |
| NM_012290 | chr2 | 171843202 | 171844410 | TLK1 | • | | |
| NM_001193528 | chr2 | 175086383 | 175086752 | SCRN3 | • | | |
| NR_026966 | chr2 | 178082238 | 178082772 | LOC100130691 | • | | |
| NM_001128144 | chr2 | 190472769 | 190473769 | PMS1 | • | | |
| NM_199440 | chr2 | 198191088 | 198191715 | HSPD1 | • | | |
| NM_021824 | chr2 | 201579689 | 201580307 | NIF3L1 | • | | |
| NM_018571 | chr2 | 202142471 | 202143236 | STRADB | • | | |
| NM_173511 | chr2 | 203323728 | 203324851 | FAM117B | • | | |
| NM_003872 | chr2 | 206471042 | 206471328 | NRP2 | • | | |
| NM_003936 | chr2 | 219649600 | 219649942 | CDK5R2 | • | | |
| NM_001927 | chr2 | 220107967 | 220108628 | DES | • | | |
| NM_005876 | chr2 | 220133043 | 220133339 | SPEG | • | | |
| NM_001195731 | chr2 | 220232048 | 220232257 | CHPF | • | | |
| NM_030768 | chr2 | 238894518 | 238894783 | ILKAP | • | | |
| NM_014674 | chr3 | 5202091 | 5202731 | EDEM1 | • | | |
| NM_024923 | chr3 | 13437731 | 13439205 | NUP210 | • | | |
| NM_014805 | chr3 | 37009703 | 37010176 | EPM2AIP1 | • | | |
| NM_002078 | chr3 | 37258709 | 37258922 | GOLGA4 | • | | |
| NM_002078 | chr3 | 37258923 | 37259567 | GOLGA4 | • | | |
| NM_005109 | chr3 | 38180936 | 38181208 | OXSR1 | • | | |
| NM_207404 | chr3 | 42921776 | 42922501 | ZNF662 | • | | |
| NM_001130082 | chr3 | 48446526 | 48447281 | PLXNB1 | • | | |
| NR_028435 | chr3 | 49434986 | 49436378 | AMT | • | | |
| NM_001640 | chr3 | 49685519 | 49685997 | APEH | • | | |
| NM_020676 | chr3 | 58197200 | 58197750 | ABHD6 | • | | |
| NM_001173468 | chr3 | 58394724 | 58395569 | PDHB | • | | |
| NR_038283 | chr3 | 62278537 | 62279717 | LOC100506994 | • | | |
| NM_025075 | chr3 | 63823453 | 63823803 | THOC7 | • | | |
| NM_007114 | chr3 | 69184353 | 69184877 | TMF1 | • | | |
| NM_001003794 | chr3 | 129025461 | 129026251 | MGLL | • | | |
| NM_153330 | chr3 | 129668727 | 129669499 | DNAJB8 | • | | |
| NM_002950 | chr3 | 129817604 | 129818298 | RPN1 | • | | |
| NM_020741 | chr3 | 130175121 | 130175578 | KIAA1257 | • | | |
| NM_001870 | chr3 | 150063953 | 150064397 | CPA3 | • | | |
| NM_002888 | chr3 | 159933151 | 159933495 | RARRES1 | • | | |
| NM_021629 | chr3 | 180652291 | 180652963 | GNB4 | • | | |
| NM_181573 | chr3 | 188007343 | 188007772 | RFC4 | • | | |
| NM_181573 | chr3 | 188007773 | 188008145 | RFC4 | • | | |
| NM_018192 | chr3 | 191321560 | 191321816 | LEPREL1 | • | | |
| NM_012287 | chr3 | 196645326 | 196646205 | ACAP2 | • | | |
| NM_152672 | chr3 | 197430169 | 197430607 | OSTalpha | • | | |
| NM_001042540 | chr3 | 198156367 | 198156965 | NCBP2 | • | | |
| NM_005929 | chr3 | 198245645 | 198247002 | MFI2 | • | | |
| NM_007100 | chr4 | 658938 | 659320 | ATP5I | • | | |
| NM_002938 | chr4 | 2436560 | 2436866 | RNF4 | • | | |
| NM_182982 | chr4 | 3003047 | 3004265 | GRK4 | • | | |
| NM_182982 | chr4 | 3004266 | 3004866 | GRK4 | • | | |
| NM_001528 | chr4 | 3478532 | 3478999 | HGFAC | • | | |
| NM_001014809 | chr4 | 6021445 | 6022370 | CRMP1 | • | | |
| NM_020773 | chr4 | 7030651 | 7030866 | TBC1D14 | • | | |
| NR_026804 | chr4 | 38499868 | 38500348 | FLJ13197 | • | | |
| NM_152398 | chr4 | 48749869 | 48750262 | OCIAD2 | • | | |
| NM_018243 | chr4 | 78226311 | 78226619 | SEP11′ | • | | |
| NM_001201 | chr4 | 82307339 | 82308340 | BMP3 | • | | |
| NM_133636 | chr4 | 84732902 | 84733773 | HELQ | • | | |
| NM_006726 | chr4 | 152294680 | 152295602 | LRBA | • | | |
| NM_015196 | chr4 | 154743345 | 154744439 | KIAA0922 | • | | |
| NM_000857 | chr4 | 157037093 | 157037800 | GUCY1B3 | • | | |
| NM_182662 | chr4 | 171386523 | 171387074 | AADAT | • | | |
| NM_152295 | chr5 | 33475064 | 33476298 | TARS | | • | |
| NR_034085 | chr5 | 43054155 | 43054431 | LOC648987 | • | | |
| NM_198566 | chr5 | 43551071 | 43552189 | C5orf34 | • | | |
| NM_181523 | chr5 | 67557695 | 67558467 | PIK3R1 | • | | |
| NM_004607 | chr5 | 77108121 | 77108484 | TBCA | • | | |
| NM_001867 | chr5 | 85948785 | 85949343 | COX7C | • | | |
| NM_014639 | chr5 | 94916312 | 94916707 | TTC37 | • | | |
| NM_003337 | chr5 | 133734172 | 133734451 | UBE2B | • | | |
| NM_024715 | chr5 | 134236169 | 134237110 | TXNDC15 | • | | |
| NM_001945 | chr5 | 139706700 | 139707349 | HBEGF | | • | |
| NM_022481 | chr5 | 141040128 | 141040897 | ARAP3 | • | | |
| NM_030571 | chr5 | 141468045 | 141468407 | NDFIP1 | • | | |
| NR_029684 | chr5 | 148781648 | 148782221 | MIR143 | • | | |
| NM_000405 | chr5 | 150611587 | 150612407 | GM2A | • | | |
| NM_017838 | chr5 | 177509311 | 177509698 | NHP2 | • | | |
| NM_139068 | chr5 | 179638857 | 179640249 | MAPK9 | | • | |
| NM_152547 | chr5 | 180398397 | 180399118 | BTNL9 | • | | |
| NR_026856 | chr6 | 2933036 | 2933918 | DKFZP686I15217 | • | | |
| NM_000904 | chr6 | 2969926 | 2970589 | NQO2 | • | | |
| NM_152551 | chr6 | 7533766 | 7535367 | SNRNP48 | • | | |
| NM_001242827 | chr6 | 13681759 | 13682012 | SIRT5 | • | | |
| NM_001143942 | chr6 | 17389178 | 17389461 | RBM24 | • | | |
| NM_001080 | chr6 | 24601607 | 24602784 | ALDH5A1 | • | | |
| NM_003495 | chr6 | 27213765 | 27214980 | HIST1H4I | • | | |
| NM_021959 | chr6 | 30142976 | 30144351 | PPP1R11 | • | | |
| NM_014641 | chr6 | 30792263 | 30792967 | MDC1 | | • | |
| NM_000594 | chr6 | 31650719 | 31651518 | TNF | • | | |
| NR_003673 | chr6 | 31790002 | 31791053 | LY6G6E | | • | |
| NM_002904 | chr6 | 32034918 | 32035873 | RDBP | • | | |
| NM_052961 | chr6 | 36100387 | 36100668 | SLC26A8 | • | | |
| NM_052961 | chr6 | 36100769 | 36101140 | SLC26A8 | • | | |
| NM_001220778 | chr6 | 36773218 | 36774114 | CDKN1A | • | | |
| NM_002648 | chr6 | 37245432 | 37245710 | PIM1 | • | | |
| NM_145316 | chr6 | 37332773 | 37333050 | TMEM217 | • | | |
| NM_024807 | chr6 | 41276999 | 41277895 | TREML2 | • | | |
| NM_018426 | chr6 | 44204401 | 44204938 | TMEM63B | • | | |
| NR_039790 | chr6 | 44331285 | 44332014 | MIR4647 | • | | hsa-mir-4647 |
| NM_178148 | chr6 | 44332015 | 44332294 | SLC35B2 | • | | |
| NM_020745 | chr6 | 44389200 | 44390430 | AARS2 | | • | |
| NM_018100 | chr6 | 52375697 | 52376108 | EFHC1 | • | | |
| NM_018100 | chr6 | 52376129 | 52376489 | EFHC1 | • | | |
| NM_145267 | chr6 | 71332584 | 71333192 | C6orf57 | • | | |
| NM_138409 | chr6 | 84799717 | 84799975 | MRAP2 | • | | |
| NM_005068 | chr6 | 101018837 | 101019121 | SIM1 | • | | |
| NM_145315 | chr6 | 108722123 | 108722667 | LACE1 | • | | |
| NM_001016 | chr6 | 133177380 | 133177628 | RPS12 | • | | |
| NM_014892 | chr6 | 155144933 | 155146299 | SCAF8 | • | | |
| NM_138810 | chr6 | 159436504 | 159437411 | TAGAP | • | | |
| NM_001080453 | chr7 | 1318807 | 1319045 | INTS1 | • | | |
| NM_032302 | chr7 | 1381984 | 1382393 | PSMG3 | • | | |
| NM_014855 | chr7 | 4586839 | 4587756 | KIAA0415 | • | | |
| NM_006854 | chr7 | 6296089 | 6296304 | KDELR2 | • | | |
| NM_018951 | chr7 | 26987900 | 26988141 | HOXA10 | • | | |
| NM_175061 | chr7 | 27994083 | 27994906 | JAZF1 | • | | |
| NM_031311 | chr7 | 28959335 | 28960180 | CPVL | • | | |
| NM_133468 | chr7 | 33537855 | 33538648 | BMPER | • | | |
| NM_031267 | chr7 | 39762911 | 39763150 | CDK13 | • | | |
| NM_014146 | chr7 | 73068288 | 73068798 | LAT2 | • | | |
| NM_003227 | chr7 | 99883967 | 99884282 | TFR2 | • | | |
| NM_003227 | chr7 | 99884283 | 99884613 | TFR2 | • | | |
| NM_017621 | chr7 | 101685411 | 101685949 | ALKBH4 | • | | |
| NM_024814 | chr7 | 106976659 | 106977608 | CBLL1 | • | | |
| NM_001201372 | chr7 | 128024369 | 128024576 | CCDC136 | • | | |
| NM_182697 | chr7 | 129186875 | 129187462 | UBE2H | • | | |
| NM_032842 | chr7 | 129439265 | 129439782 | TMEM209 | • | | |
| NM_014690 | chr7 | 142575908 | 142576122 | FAM131B | • | | |
| NM_001099220 | chr7 | 148980237 | 148980824 | ZNF862 | • | | |
| NM_001091 | chr7 | 149986937 | 149987454 | ABP1 | • | | |
| NM_005542 | chr7 | 154525734 | 154526238 | INSIG1 | • | | |
| NM_018941 | chr8 | 1697917 | 1698652 | CLN8 | • | | |
| NM_001007090 | chr8 | 13468790 | 13470190 | C8orf48 | | • | |
| NM_054026 | chr8 | 17147581 | 17148794 | CNOT7 | • | | |
| NM_004686 | chr8 | 17250882 | 17251456 | MTMR7 | • | | |
| NM_025151 | chr8 | 37877091 | 37877986 | RAB11FIP1 | • | | |
| NM_004198 | chr8 | 42743049 | 42743635 | CHRNA6 | • | | |
| NM_001023 | chr8 | 57149697 | 57150240 | RPS20 | • | | |
| NM_003878 | chr8 | 64114680 | 64115167 | GGH | • | | |
| NM_019607 | chr8 | 67742243 | 67743259 | C8orf44 | • | | |
| NM_170709 | chr8 | 67848775 | 67849399 | SGK3 | • | | |
| NM_004337 | chr8 | 90982606 | 90983258 | OSGIN2 | • | | |
| NM_152628 | chr8 | 101731190 | 101732002 | SNX31 | • | | |
| NM_022783 | chr8 | 120954692 | 120955088 | DEPTOR | • | | |
| NM_207006 | chr8 | 124263484 | 124263939 | FAM83A | • | | |
| NM_194291 | chr8 | 125454577 | 125455045 | TMEM65 | • | | |
| NR_040712 | chr8 | 141649045 | 141649431 | CHRAC1 | • | | |
| NM_024736 | chr8 | 144710336 | 144711366 | GSDMD | • | | |
| NM_017767 | chr8 | 145610684 | 145611022 | SLC39A4 | • | | |
| NM_001039697 | chr9 | 15412256 | 15412569 | SNAPC3 | • | | |
| NM_003026 | chr9 | 17568881 | 17569127 | SH3GL2 | • | | |
| NM_018449 | chr9 | 34039406 | 34040261 | UBAP2 | • | | |
| NM_147169 | chr9 | 34371892 | 34372298 | C9orf24 | | • | |
| NM_014450 | chr9 | 35640953 | 35641984 | SIT1 | • | | |
| NM_194330 | chr9 | 36391255 | 36391616 | RNF38 | | • | |
| NM_001135820 | chr9 | 71614390 | 71615744 | TMEM2 | | • | |
| NM_030940 | chr9 | 86127331 | 86128205 | ISCA1 | • | | |
| NM_032012 | chr9 | 108961697 | 108963419 | C9orf5 | • | | |
| NM_015651 | chr9 | 120732830 | 120733244 | PHF19 | • | | |
| NM_005347 | chr9 | 125083916 | 125084532 | HSPA5 | • | | |
| NM_001012502 | chr9 | 127552463 | 127553128 | C9orf117 | • | | |
| NM_000476 | chr9 | 127719676 | 127720136 | AK1 | • | | |
| NM_001134707 | chr9 | 133596668 | 133596902 | SARDH | • | | |
| NM_015447 | chr9 | 136000683 | 136001323 | CAMSAP1 | • | | |
| NM_181701 | chr9 | 136345003 | 136345693 | QSOX2 | • | | |
| NM_016215 | chr9 | 136832457 | 136832694 | EGFL7 | • | | |
| NR_024580 | chr9 | 136979041 | 136979655 | LOC100131193 | • | | |
| NM_013379 | chr9 | 137285554 | 137285938 | DPP7 | • | | |
| NM_013379 | chr9 | 137285939 | 137286154 | DPP7 | • | | |
| NM_006088 | chr9 | 137412572 | 137412972 | TUBB4B | • | | |
| NM_001004354 | chr9 | 137473820 | 137474042 | NRARP | • | | |
| NM_033261 | chr10 | 1060430 | 1061901 | IDI2 | • | | |
| NM_205845 | chr10 | 5052184 | 5052700 | AKR1C2 | | • | |
| NM_152751 | chr10 | 13584869 | 13586369 | BEND7 | • | | |
| NR_024284 | chr10 | 31646819 | 31647269 | ZEB1-AS1 | • | | |
| NM_001198777 | chr10 | 35419566 | 35420138 | CUL2 | • | | |
| NM_001098204 | chr10 | 43225489 | 43226005 | HNRNPF | • | | |
| NM_032023 | chr10 | 44775263 | 44775563 | RASSF4 | • | | |
| NM_020999 | chr10 | 71002980 | 71003225 | NEUROG3 | • | | |
| NM_001083116 | chr10 | 72032624 | 72032882 | PRF1 | • | | |
| NM_001083116 | chr10 | 72032883 | 72033277 | PRF1 | • | | |
| NM_152710 | chr10 | 72203735 | 72204133 | C10orf27 | • | | |
| NM_152710 | chr10 | 72214392 | 72215683 | C10orf27 | • | | |
| NM_138357 | chr10 | 74120329 | 74120939 | MCU | • | | |
| NM_032810 | chr10 | 89566291 | 89567467 | ATAD1 | • | | |
| NM_001114094 | chr10 | 98020200 | 98021250 | BLNK | | • | |
| NM_012215 | chr10 | 103568439 | 103569550 | MGEA5 | • | | |
| NM_152310 | chr10 | 103974705 | 103976068 | ELOVL3 | | • | |
| NM_145206 | chr10 | 114197707 | 114198508 | VTI1A | • | | |
| NM_173791 | chr10 | 119125739 | 119126231 | PDZD8 | • | | |
| NM_001005339 | chr10 | 121293107 | 121293502 | RGS10 | • | | |
| NR_038365 | chr10 | 133474386 | 133474793 | FLJ46300 | • | | |
| NM_006659 | chr10 | 135011902 | 135012124 | TUBGCP2 | • | | |
| NM_203383 | chr11 | 493466 | 494069 | RNH1 | • | | |
| NM_003475 | chr11 | 551919 | 552261 | RASSF7 | • | | |
| NM_001142677 | chr11 | 901693 | 902453 | CHID1 | • | | |
| NM_002339 | chr11 | 1829780 | 1830345 | LSP1 | • | | |
| NM_007105 | chr11 | 2881649 | 2882045 | SLC22A18AS | • | | |
| NM_001164377 | chr11 | 3196789 | 3197099 | MRGPRG | • | | |
| NM_001143976 | chr11 | 9552980 | 9553299 | WEE1 | • | | |
| NM_001202439 | chr11 | 17328938 | 17329683 | B7H6 | • | | |
| NM_001256372 | chr11 | 19220849 | 19221328 | E2F8 | • | | |
| NM_018490 | chr11 | 27450997 | 27451964 | LGR4 | • | | |
| NM_001206615 | chr11 | 34598501 | 34599147 | EHF | • | | |
| NM_024841 | chr11 | 36353285 | 36354143 | PRR5L | • | | |
| NM_000256 | chr11 | 47330763 | 47331342 | MYBPC3 | • | | |
| NM_003146 | chr11 | 56860067 | 56861058 | SSRP1 | • | | |
| NM_024098 | chr11 | 60364867 | 60365771 | CCDC86 | • | | |
| NM_004778 | chr11 | 60380143 | 60381551 | PTGDR2 | • | | |
| NM_017966 | chr11 | 60685779 | 60686763 | VPS37C | • | | |
| NM_013401 | chr11 | 61441954 | 61443206 | RAB3IL1 | • | | |
| NM_013401 | chr11 | 61443322 | 61444278 | RAB3IL1 | • | | |
| NM_004739 | chr11 | 62127390 | 62128171 | MTA2 | • | | |
| NM_198335 | chr11 | 62173406 | 62173726 | GANAB | • | | |
| NM_024099 | chr11 | 62196280 | 62196683 | C11orf48 | • | | |
| NM_001130702 | chr11 | 62230531 | 62231168 | BSCL2 | • | | |
| NM_017878 | chr11 | 63088397 | 63088787 | HRASLS2 | • | | |
| NM_006795 | chr11 | 64403383 | 64403704 | EHD1 | • | | |
| NR_037650 | chr11 | 64536912 | 64537751 | ARL2-SNX15 | • | | |
| NM_172230 | chr11 | 64659306 | 64659976 | SYVN1 | • | | |
| NM_002689 | chr11 | 64784716 | 64785758 | POLA2 | • | | |
| NR_038923 | chr11 | 65094040 | 65094271 | LOC254100 | • | | |
| NM_032223 | chr11 | 65149874 | 65150260 | PCNXL3 | • | | |
| NM_002869 | chr11 | 73149903 | 73150216 | RAB6A | • | | |
| NM_003355 | chr11 | 73372563 | 73372934 | UCP2 | • | | |
| NM_004055 | chr11 | 76453319 | 76454972 | CAPN5 | • | | |
| NM_016156 | chr11 | 95297233 | 95298255 | MTMR2 | • | | |
| NM_001931 | chr11 | 111399727 | 111400844 | DLAT | • | | |
| NM_003904 | chr11 | 116164347 | 116164731 | ZNF259 | • | | |
| NM_015157 | chr11 | 117982244 | 117983525 | PHLDB1 | • | | |
| NM_001164280 | chr11 | 118405547 | 118405872 | SLC37A4 | • | | |
| NM_001164280 | chr11 | 118405873 | 118406215 | SLC37A4 | • | | |
| NM_024618 | chr11 | 118543262 | 118544312 | NLRX1 | | • | |
| NM_020716 | chr11 | 122935027 | 122935933 | GRAMD1B | • | | |
| NM_019055 | chr11 | 124272991 | 124274023 | ROBO4 | | • | |
| NM_000890 | chr11 | 128265119 | 128265470 | KCNJ5 | • | | |
| NM_001039920 | chr12 | 6669182 | 6670204 | ZNF384 | | • | |
| NR_026581 | chr12 | 6733229 | 6734509 | MLF2 | | • | |
| NM_031491 | chr12 | 7172761 | 7173347 | RBP5 | • | | |
| NM_031491 | chr12 | 7173348 | 7173587 | RBP5 | • | | |
| NM_033360 | chr12 | 25295755 | 25296119 | KRAS | • | | |
| NM_016594 | chr12 | 47605688 | 47606434 | FKBP11 | • | | |
| NM_002733 | chr12 | 47698992 | 47700528 | PRKAG1 | • | | |
| NM_002733 | chr12 | 47700728 | 47701180 | PRKAG1 | • | | |
| NM_005276 | chr12 | 48782682 | 48783104 | GPD1 | • | | |
| NM_175078 | chr12 | 51383591 | 51384331 | KRT77 | • | | |
| NM_002624 | chr12 | 51974958 | 51975494 | PFDN5 | • | | |
| NM_006163 | chr12 | 52977641 | 52978040 | NFE2 | • | | |
| NM_020370 | chr12 | 53044631 | 53045562 | GPR84 | • | | |
| NM_001172696 | chr12 | 56461270 | 56462726 | TSFM | • | | |
| NM_178169 | chr12 | 63306017 | 63306302 | RASSF3 | • | | |
| NM_007007 | chr12 | 67919239 | 67919449 | CPSF6 | • | | |
| NM_006654 | chr12 | 68149530 | 68150140 | FRS2 | • | | |
| NM_024685 | chr12 | 75243205 | 75244377 | BBS10 | • | | |
| NM_002635 | chr12 | 97488891 | 97489709 | SLC25A3 | • | | |
| NM_001031701 | chr12 | 102737653 | 102737867 | NT5DC3 | | • | |
| NM_014055 | chr12 | 109023675 | 109024689 | IFT81 | • | | |
| NM_006768 | chr12 | 110585488 | 110586156 | BRAP | • | | |
| NM_019034 | chr12 | 120698011 | 120698331 | RHOF | • | | |
| NM_198240 | chr12 | 121432682 | 121433683 | CLIP1 | | • | |
| NR_027918 | chr12 | 121783282 | 121783755 | CCDC62 | • | | |
| NM_021009 | chr12 | 123922831 | 123923352 | UBC | • | | hsa-mir-5188 |
| NM_000059 | chr13 | 31786963 | 31787560 | BRCA2 | • | | |
| NM_203451 | chr13 | 36145136 | 36145506 | SERTM1 | • | | |
| NM_002298 | chr13 | 45656100 | 45656488 | LCP1 | • | | |
| NR_037407 | chr13 | 49467453 | 49468385 | MIR3613 | | • | hsa-mir-3613 |
| NM_080759 | chr13 | 71339613 | 71340674 | DACH1 | • | | |
| NM_021033 | chr13 | 96883693 | 96884286 | RAP2A | • | | |
| NM_003576 | chr13 | 97880215 | 97881366 | STK24 | | • | |
| NM_138779 | chr13 | 102224930 | 102225703 | TEX30 | • | | |
| NM_198235 | chr14 | 20340858 | 20341311 | RNASE1 | • | | |
| NM_002471 | chr14 | 22948432 | 22949129 | MYH6 | | • | |
| NM_004554 | chr14 | 23906682 | 23907154 | NFATC4 | • | | |
| NM_003082 | chr14 | 61297915 | 61298758 | SNAPC1 | • | | |
| NM_182526 | chr14 | 67052243 | 67052773 | TMEM229B | • | | |
| NM_001244701 | chr14 | 68333328 | 68333858 | ZFP36L1 | • | | |
| NM_194279 | chr14 | 74030796 | 74031074 | ISCA2 | • | | |
| NM_194279 | chr14 | 74038035 | 74038316 | ISCA2 | • | | |
| NR_003709 | chr14 | 90681087 | 90681784 | SNORA11B | • | | |
| NM_032490 | chr14 | 92744133 | 92744831 | C14orf142 | • | | |
| NM_018036 | chr14 | 95900365 | 95901640 | ATG2B | • | | |
| NM_002376 | chr14 | 102920112 | 102920884 | MARKS | | • | |
| NM_152328 | chr14 | 104255983 | 104256280 | ADSSL1 | • | | |
| NM_138790 | chr14 | 104477170 | 104477566 | PLD4 | • | | |
| NM_015995 | chr15 | 29404425 | 29404998 | KLF13 | • | | |
| NM_170589 | chr15 | 38671826 | 38672819 | CASC5 | | • | |
| NM_170589 | chr15 | 38672820 | 38673397 | CASC5 | • | | |
| NM_001159629 | chr15 | 48260539 | 48261614 | SLC27A2 | • | | |
| NM_153374 | chr15 | 49817403 | 49818168 | LYSMD2 | | • | |
| NM_194272 | chr15 | 62855981 | 62857302 | RBPMS2 | • | | |
| NM_006049 | chr15 | 64577266 | 64577476 | SNAPC5 | • | | hsa-mir-4512 |
| NM_001099436 | chr15 | 72922872 | 72923285 | ULK3 | • | | |
| NM_001127716 | chr15 | 74391035 | 74391426 | ETFA | • | | |
| NM_003978 | chr15 | 75073618 | 75073940 | PSTPIP1 | • | | |
| NM_003978 | chr15 | 75074036 | 75074506 | PSTPIP1 | • | | |
| NM_001256567 | chr15 | 76720614 | 76721399 | CHRNB4 | • | | |
| NM_001256567 | chr15 | 76721400 | 76722304 | CHRNB4 | • | | |
| NM_000137 | chr15 | 78233506 | 78234312 | FAH | • | | |
| NM_001008226 | chr15 | 80342577 | 80343558 | FAM154B | • | | |
| NM_001011885 | chr15 | 81527552 | 81527841 | BTBD1 | • | | hsa-mir-4515 |
| NM_020212 | chr15 | 88035261 | 88035675 | WDR93 | • | | |
| NM_020210 | chr15 | 88528406 | 88528830 | SEMA4B | • | | |
| NM_001143785 | chr15 | 89227268 | 89227545 | FES | • | | |
| NM_022450 | chr16 | 63444 | 63915 | RHBDF1 | • | | |
| NM_006849 | chr16 | 272083 | 272382 | PDIA2 | • | | |
| NM_176677 | chr16 | 556363 | 556827 | NHLRC4 | • | | |
| NM_004204 | chr16 | 558795 | 559610 | PIGQ | • | | |
| NM_001005920 | chr16 | 675195 | 675526 | JMJD8 | • | | |
| NM_001010865 | chr16 | 1764597 | 1765093 | EME2 | • | | |
| NM_005061 | chr16 | 1944922 | 1945333 | RPL3L | • | | |
| NM_080594 | chr16 | 2259437 | 2259665 | RNPS1 | • | | hsa-mir-3677 |
| NM_015944 | chr16 | 2509965 | 2510169 | AMDHD2 | • | | |
| NM_016333 | chr16 | 2741166 | 2741922 | SRRM2 | • | | |
| NM_001103175 | chr16 | 3022094 | 3022544 | CCDC64B | • | | |
| NM_138440 | chr16 | 4360292 | 4360865 | VASN | • | | |
| NM_024109 | chr16 | 8645499 | 8646609 | METTL22 | • | | |
| NM_001802 | chr16 | 22293930 | 22294882 | CDR2 | • | | |
| NR_002453 | chr16 | 30254623 | 30254833 | LOC595101 | • | | |
| NM_152652 | chr16 | 30316799 | 30317011 | ZNF48 | • | | |
| NM_152652 | chr16 | 30317157 | 30317590 | ZNF48 | • | | |
| NM_024031 | chr16 | 30568833 | 30569069 | PRR14 | • | | |
| NM_022744 | chr16 | 31427385 | 31428655 | C16orf58 | • | | |
| NM_000293 | chr16 | 46052754 | 46053098 | PHKB | • | | |
| NR_026889 | chr16 | 54786025 | 54787339 | DKFZP434H168 | | • | |
| NM_000339 | chr16 | 55456229 | 55456506 | SLC12A3 | • | | |
| NM_002987 | chr16 | 55995879 | 55997083 | CCL17 | | • | |
| NM_005550 | chr16 | 56394059 | 56394614 | KIFC3 | • | | |
| NM_006565 | chr16 | 66152412 | 66153241 | CTCF | • | | |
| NM_020850 | chr16 | 66371560 | 66372539 | RANBP10 | • | | |
| NM_020850 | chr16 | 66372540 | 66372822 | RANBP10 | • | | |
| NM_138383 | chr16 | 69253098 | 69253368 | MTSS1L | • | | |
| NM_001030007 | chr16 | 70400943 | 70401381 | AP1G1 | • | | |
| NM_032268 | chr16 | 73589452 | 73589908 | ZNRF1 | • | | |
| NM_021197 | chr16 | 82914283 | 82914948 | WFDC1 | • | | |
| NM_014732 | chr16 | 83653877 | 83654132 | KIAA0513 | • | | |
| NM_198491 | chr16 | 83704767 | 83705133 | FAM92B | | • | |
| NM_001159380 | chr16 | 85144751 | 85145959 | MTHFSD | • | | |
| NM_002768 | chr16 | 88250462 | 88250864 | CHMP1A | • | | |
| NM_015721 | chr17 | 600353 | 601081 | GEMIN4 | • | | |
| NM_018146 | chr17 | 631205 | 631803 | RNMTL1 | • | | |
| NM_021947 | chr17 | 2154773 | 2155142 | SRR | • | | |
| NM_001100398 | chr17 | 2645217 | 2646254 | RAP1GAP2 | • | | |
| NM_031965 | chr17 | 3572498 | 3573624 | GSG2 | • | | |
| NM_002208 | chr17 | 3652514 | 3653239 | ITGAE | • | | |
| NM_014520 | chr17 | 4406157 | 4406978 | MYBBP1A | • | | |
| NR_002912 | chr17 | 7421686 | 7422153 | SNORA67 | • | | |
| NM_012393 | chr17 | 8092931 | 8093243 | PFAS | • | | |
| NM_001004313 | chr17 | 10575097 | 10575322 | TMEM220 | • | | |
| NM_144775 | chr17 | 18159975 | 18161334 | SMCR8 | | • | |
| NM_016231 | chr17 | 23392040 | 23392713 | NLK | • | | |
| NM_001242366 | chr17 | 23756684 | 23757187 | SLC46A1 | • | | |
| NM_005148 | chr17 | 23903869 | 23905128 | UNC119 | • | | |
| NM_024857 | chr17 | 26182985 | 26183196 | ATAD5 | • | | |
| NM_138328 | chr17 | 27615581 | 27616202 | RHBDL3 | | • | |
| NM_001163545 | chr17 | 33043702 | 33044444 | SYNRG | • | | |
| NM_001024809 | chr17 | 35760339 | 35760792 | RARA | • | | |
| NM_000422 | chr17 | 37034362 | 37034807 | KRT17 | • | | |
| NM_001252039 | chr17 | 37561140 | 37561428 | RAB5C | • | | |
| NM_003152 | chr17 | 37691485 | 37692036 | STAT5A | • | | |
| NM_001099225 | chr17 | 40122778 | 40123381 | CCDC43 | • | | |
| NM_001242376 | chr17 | 40348450 | 40349253 | GFAP | • | | |
| NM_021079 | chr17 | 40494547 | 40495804 | NMT1 | • | | |
| NM_152343 | chr17 | 40693944 | 40694983 | TEX34 | • | | |
| NM_001002841 | chr17 | 42641441 | 42642472 | MYL4 | • | | |
| NM_001112707 | chr17 | 57908294 | 57908830 | TLK2 | • | | |
| NM_001098426 | chr17 | 59274020 | 59274347 | SMARCD2 | • | | |
| NM_000442 | chr17 | 59787488 | 59788289 | PECAM1 | • | | |
| NM_001545 | chr17 | 70519276 | 70519909 | ICT1 | • | | |
| NR_036520 | chr17 | 70779572 | 70779902 | LOC100287042 | • | | |
| NM_001258 | chr17 | 71509068 | 71509383 | CDK3 | • | | |
| NM_015219 | chr17 | 71611468 | 71611926 | EXOC7 | • | | |
| NR_040050 | chr17 | 73068311 | 73068696 | LOC100507351 | • | | |
| NM_001163075 | chr17 | 73652919 | 73653170 | C17orf99 | • | | |
| NM_003258 | chr17 | 73694869 | 73695483 | TK1 | • | | |
| NM_003255 | chr17 | 74441468 | 74441689 | TIMP2 | • | | |
| NM_001082575 | chr17 | 74901770 | 74902146 | RBFOX3 | • | | |
| NM_178520 | chr17 | 76920047 | 76920387 | TMEM105 | • | | |
| NM_001206950 | chr17 | 77778624 | 77779240 | SLC16A3 | • | | |
| NM_032142 | chr18 | 12996584 | 12996984 | CEP192 | • | | |
| NM_000140 | chr18 | 53405090 | 53406605 | FECH | • | | |
| NM_001168499 | chr18 | 70326383 | 70327093 | CNDP2 | • | | |
| NM_032510 | chr18 | 76107020 | 76108260 | PARD6G | • | | |
| NM_004359 | chr19 | 481965 | 482307 | CDC34 | • | | |
| NM_005224 | chr19 | 879464 | 879945 | ARID3A | • | | |
| NM_002695 | chr19 | 1046289 | 1047195 | POLR2E | • | | |
| NM_000455 | chr19 | 1155937 | 1156406 | STK11 | | • | |
| NM_004152 | chr19 | 2219397 | 2220089 | OAZ1 | • | | |
| NM_198532 | chr19 | 2233726 | 2234123 | C19orf35 | • | | |
| NM_021938 | chr19 | 3174335 | 3175395 | CELF5 | • | | |
| NM_001171091 | chr19 | 4406413 | 4406736 | UBXN6 | • | | |
| NM_001171091 | chr19 | 4406737 | 4407622 | UBXN6 | • | | |
| NM_130855 | chr19 | 5238303 | 5238734 | PTPRS | • | | |
| NM_001042462 | chr19 | 7650799 | 7651612 | TRAPPC5 | • | | |
| NM_145245 | chr19 | 7800750 | 7801132 | EVI5L | • | | |
| NM_016579 | chr19 | 8279194 | 8279825 | CD320 | • | | |
| NM_022377 | chr19 | 10256085 | 10256286 | ICAM4 | • | | |
| NM_022377 | chr19 | 10256489 | 10256714 | ICAM4 | • | | |
| NM_004283 | chr19 | 11293630 | 11293898 | RAB3D | • | | |
| NM_001164276 | chr19 | 12266873 | 12267626 | ZNF44 | • | | |
| NM_006563 | chr19 | 12858892 | 12859512 | KLF1 | • | | |
| NM_024074 | chr19 | 16632653 | 16632863 | TMEM38A | • | | |
| NM_001238 | chr19 | 34993294 | 34993869 | CCNE1 | • | | |
| NM_032139 | chr19 | 37856542 | 37857254 | ANKRD27 | • | | |
| NM_004364 | chr19 | 38483180 | 38483826 | CEBPA | • | | |
| NR_026887 | chr19 | 38487407 | 38488023 | LOC80054 | • | | |
| NM_022835 | chr19 | 44606513 | 44607157 | PLEKHG2 | • | | |
| NM_004756 | chr19 | 45889140 | 45889364 | NUMBL | • | | |
| NR_038452 | chr19 | 52680298 | 52681145 | LOC100505681 | • | | |
| NM_000836 | chr19 | 53589173 | 53589609 | GRIN2D | • | | |
| NM_020904 | chr19 | 54063741 | 54064493 | PLEKHA4 | • | | |
| NM_144688 | chr19 | 54582914 | 54583202 | CCDC155 | • | | |
| NM_030973 | chr19 | 55026822 | 55027257 | MED25 | • | | hsa-mir-6800 |
| NM_030973 | chr19 | 55027398 | 55027930 | MED25 | • | | hsa-mir-6800 |
| NR_039904 | chr19 | 55049812 | 55050059 | MIR4749 | • | | hsa-mir-4749 |
| NM_001193357 | chr19 | 55124647 | 55125750 | NUP62 | • | | |
| NM_138411 | chr19 | 55672193 | 55674042 | FAM71E1 | | • | |
| NM_000363 | chr19 | 60361054 | 60362099 | TNNI3 | • | | |
| NM_000363 | chr19 | 60362100 | 60362453 | TNNI3 | • | | |
| NM_153219 | chr19 | 60804114 | 60805331 | ZNF524 | • | | |
| NM_001130071 | chr19 | 60898111 | 60898493 | EPN1 | • | | |
| NM_001085384 | chr19 | 62912968 | 62913576 | ZNF154 | • | | |
| NM_080725 | chr20 | 582329 | 583546 | SRXN1 | | • | |
| NM_153640 | chr20 | 3820110 | 3820425 | PANK2 | • | | |
| NR_034149 | chr20 | 23061060 | 23062405 | LOC200261 | • | | |
| NM_002657 | chr20 | 30257498 | 30258756 | PLAGL2 | • | | |
| NM_005225 | chr20 | 31738132 | 31738671 | E2F1 | • | | |
| NM_198398 | chr20 | 33593430 | 33594012 | ERGIC3 | • | | |
| NM_015511 | chr20 | 34286713 | 34287463 | C20orf4 | • | | |
| NM_199181 | chr20 | 34923272 | 34923618 | KIAA0889 | • | | |
| NM_198941 | chr20 | 42584174 | 42585016 | SERINC3 | • | | |
| NM_198941 | chr20 | 42585017 | 42586042 | SERINC3 | • | | |
| NM_002251 | chr20 | 43163288 | 43163988 | KCNS1 | • | | |
| NM_173073 | chr20 | 44396514 | 44398120 | SLC35C2 | | • | |
| NR_045796 | chr20 | 47094546 | 47096270 | CSE1L | • | | |
| NM_173644 | chr20 | 58063221 | 58064395 | C20orf197 | • | | |
| NR_030376 | chr20 | 58325406 | 58326778 | MIR646 | • | | |
| NM_001853 | chr20 | 60917779 | 60918058 | COL9A3 | • | | |
| NM_003823 | chr20 | 61790587 | 61790895 | TNFRSF6B | • | | |
| NR_045370 | chr20 | 61979251 | 61979462 | LOC100505815 | • | | |
| NM_006585 | chr21 | 29367756 | 29368629 | CCT8 | • | | |
| NM_000454 | chr21 | 31953394 | 31953618 | SOD1 | • | | |
| NM_001122607 | chr21 | 35185118 | 35185524 | RUNX1 | • | | |
| NM_005239 | chr21 | 39232369 | 39232934 | ETS2 | • | | |
| NM_002463 | chr21 | 41655104 | 41655865 | MX2 | • | | |
| NM_018961 | chr21 | 42696377 | 42697223 | UBASH3A | • | | |
| NM_000394 | chr21 | 43464710 | 43465021 | CRYAA | • | | |
| NM_030891 | chr21 | 44703511 | 44704210 | LRRC3 | • | | |
| NM_198688 | chr21 | 44837373 | 44838175 | KRTAP10-6 | • | | |
| NM_001163079 | chr22 | 15976851 | 15977056 | CECR6 | | • | |
| NM_173793 | chr22 | 17810394 | 17811023 | C22orf39 | • | | |
| NM_182984 | chr22 | 18478070 | 18478279 | TRMT2A | • | | hsa-mir-6816 |
| NM_058004 | chr22 | 19536332 | 19536939 | PI4KA | • | | |
| NM_001018060 | chr22 | 19637050 | 19637689 | AIFM3 | • | | |
| NM_007128 | chr22 | 20923618 | 20923926 | VPREB1 | | • | |
| NM_003073 | chr22 | 22450922 | 22452714 | SMARCB1 | • | | |
| NM_003073 | chr22 | 22453329 | 22453665 | SMARCB1 | • | | |
| NM_001037666 | chr22 | 29010612 | 29010898 | GATSL3 | • | | |
| NM_001164502 | chr22 | 30211106 | 30212410 | EIF4ENIF1 | • | | |
| NM_003405 | chr22 | 30643843 | 30644796 | YWHAH | • | | |
| NM_000362 | chr22 | 31520979 | 31522000 | TIMP3 | • | | |
| NM_002473 | chr22 | 35108932 | 35109796 | MYH9 | • | | |
| NM_001177701 | chr22 | 35497152 | 35498127 | IFT27 | • | | |
| NM_005318 | chr22 | 36524721 | 36524963 | H1F0 | • | | |
| NM_001199562 | chr22 | 36902570 | 36903013 | PLA2G6 | • | | |
| NM_001195071 | chr22 | 36987513 | 36987897 | TMEM184B | • | | |
| NM_001198726 | chr22 | 43446693 | 43447507 | ARHGAP8 | • | | |
| NM_015653 | chr22 | 44130450 | 44131360 | RIBC2 | • | | |
| NR_027033 | chr22 | 44788955 | 44789557 | MIRLET7BHG | • | | |
| NR_029479 | chr22 | 44830076 | 44830455 | MIRLET7B | • | | hsa-let-7a-3, hsa- |
| | | | | | | | let-7b, hsa-mir- |
| | | | | | | | 4763 |
| NM_006071 | chr22 | 44979792 | 44980898 | PKDREJ | • | | |
| NM_022766 | chr22 | 45455264 | 45456159 | CERK | • | | |
| NR_027691 | chr22 | 48910187 | 48910389 | PANX2 | • | | |
| NM_152299 | chr22 | 49237087 | 49238094 | NCAPH2 | • | | |
| NM_138636 | chrX | 12697137 | 12698127 | TLR8 | • | | |
| NM_006746 | chrX | 17513776 | 17515316 | SCML1 | | • | |
| NM_017883 | chrX | 48210449 | 48211728 | WDR13 | • | | |
| NM_002049 | chrX | 48400265 | 48400653 | GATA1 | • | | |
| NM_002547 | chrX | 67436878 | 67437685 | OPHN1 | • | | |
| NM_017752 | chrX | 105852209 | 105853682 | TBC1D8B | | • | |
| NM_022977 | chrX | 108782852 | 108783388 | ACSL4 | • | | |
| NM_001711 | chrX | 152280673 | 152281136 | BGN | • | | |
| NM_001139457 | chrX | 152508733 | 152510123 | BCAP31 | • | | |
| NM_001204527 | chrX | 152578988 | 152580122 | SSR4 | • | | |
| NM_001110792 | chrX | 152884156 | 152885140 | MECP2 | • | | |

### REFERENCES

1. Shih, A. H., Abdel-Wahab, O., Patel, J. P., and Levine, R. L. 2012. The role of mutations in epigenetic regulators in myeloid malignancies. Nat Rev Cancer 12:599-612.
2. Cancer Genome Atlas Research, N. 2013. Genomic and epigenomic landscapes of adult de novo acute myeloid leukemia. N Engl J Med 368:2059-2074.
3. Patel, J. P., Gonen, M., Figueroa, M. E., Fernandez, H., Sun, Z., Racevskis, J., Van Vlierberghe, P., Dolgalev, I., Thomas, S., Aminova, O., et al. 2012. Prognostic relevance of integrated genetic profiling in acute myeloid leukemia. N Engl J Med 366:1079-1089.
4. Figueroa, M. E., Lugthart, S., Li, Y., Erpelinck-Verschueren, C., Deng, X., Christos, P. J., Schifano, E., Booth, J., van Putten, W., Skrabanek, L., et al. 2010. DNA methylation signatures identify biologically distinct subtypes in acute myeloid leukemia. Cancer Cell 17:13-27.
5. Ji, H., Ehrlich, L. I., Seita, J., Murakami, P., Doi, A., Lindau, P., Lee, H., Aryee, M. J., Irizarry, R. A., Kim, K., et al. 2010. Comprehensive methylome map of lineage commitment from haematopoietic progenitors. Nature 467:338-342.
6. Shearstone, J. R., Pop, R., Bock, C., Boyle, P., Meissner, A., and Socolovsky, M. 2011. Global DNA demethylation during mouse erythropoiesis in vivo. Science 334:799-802.
7. Bock, C., Beerman, I., Lien, W. H., Smith, Z. D., Gu, H., Boyle, P., Gnirke, A., Fuchs, E., Rossi, D. J., and Meissner, A. 2012. DNA methylation dynamics during in vivo differentiation of blood and skin stem cells. Mol Cell 47:633-647.
8. Broske, A. M., Vockentanz, L., Kharazi, S., Huska, M. R., Mancini, E., Scheller, M., Kuhl, C., Enns, A., Prinz, M., Jaenisch, R., et al. 2009. DNA methylation protects hematopoietic stem cell multipotency from myeloerythroid restriction. Nat Genet 41:1207-1215.
9. Trowbridge, J. J., Snow, J. W., Kim, J., and Orkin, S. H. 2009. DNA methyltransferase 1 is essential for and uniquely regulates hematopoietic stem and progenitor cells. Cell Stem Cell 5:442-449.
10. Challen, G. A., Sun, D., Jeong, M., Luo, M., Jelinek, J., Berg, J. S., Bock, C., Vasanthakumar, A., Gu, H., Xi, Y., et al. 2012. Dnmt3a is essential for hematopoietic stem cell differentiation. Nat Genet 44:23-31.
11. Sasaki, M., Knobbe, C. B., Munger, J. C., Lind, E. F., Brenner, D., Brustle, A., Harris, I. S., Holmes, R., Wakeham, A., Haight, J., et al. 2012. IDH1(R132H) mutation increases murine haematopoietic progenitors and alters epigenetics. Nature 488:656-659.
12. Moran-Crusio, K., Reavie, L., Shih, A., Abdel-Wahab, O., Ndiaye-Lobry, D., Lobry, C., Figueroa, M. E., Vasanthakumar, A., Patel, J., Zhao, X., et al. 2011. Tet2 loss leads to increased hematopoietic stem cell self-renewal and myeloid transformation. Cancer Cell 20:11-24.
13. Quivoron, C., Couronne, L., Della Valle, V., Lopez, C. K., Plo, I., Wagner-Ballon, O., Do Cruzeiro, M., Delhommeau, F., Arnulf, B., Stern, M. H., et al. 2011. TET2 inactivation results in pleiotropic hematopoietic abnormalities in mouse and is a recurrent event during human lymphomagenesis. Cancer Cell 20:25-38.
14. Li, Z., Cai, X., Cai, C. L., Wang, J., Zhang, W., Petersen, B. E., Yang, F. C., and Xu, M. 2011. Deletion of Tet2 in mice leads to dysregulated hematopoietic stem cells and subsequent development of myeloid malignancies. Blood 118:4509-4518.
15. Baum, C. M., Weissman, I. L., Tsukamoto, A. S., Buckle, A. M., and Peault, B. 1992. Isolation of a candidate human hematopoietic stem-cell population. Proc Natl Acad Sci USA 89:2804-2808.
16. Craig, W., Kay, R., Cutler, R. L., and Lansdorp, P. M. 1993. Expression of Thy-1 on human hematopoietic progenitor cells. J Exp Med 177:1331-1342.
17. Manz, M. G., Miyamoto, T., Akashi, K., and Weissman, I. L. 2002. Prospective isolation of human clonogenic common myeloid progenitors. Proc Natl Acad Sci USA 99:11872-11877.
18. Steidl, U., Rosenbauer, F., Verhaak, R. G., Gu, X., Ebralidze, A., Otu, H. H., Klippel, S., Steidl, C., Bruns, I., Costa, D. B., et al. 2006. Essential role of Jun family transcription factors in PU.1 knockdown-induced leukemic stem cells. Nat Genet 38:1269-1277.
19. Steidl, U., Steidl, C., Ebralidze, A., Chapuy, B., Han, H. J., Will, B., Rosenbauer, F., Becker, A., Wagner, K., Koschmieder, S., et al. 2007. A distal single nucleotide polymorphism alters long-range regulation of the PU.1 gene in acute myeloid leukemia. J Clin Invest 117:2611-2620.
20. Will, B., and Steidl, U. 2010. Multi-parameter fluorescence-activated cell sorting and analysis of stem and progenitor cells in myeloid malignancies. Best Pract Res Clin Haematol 23:391-401.
21. Barreyro, L., Will, B., Bartholdy, B., Zhou, L., Todorova, T. I., Stanley, R. F., Ben-Neriah, S., Montagna, C., Parekh, S., Pellagatti, A., et al. 2012. Overexpression of IL-1 receptor accessory protein in stem and progenitor cells and outcome correlation in AML and MDS. Blood 120:1290-1298.
22. Khulan, B., Thompson, R. F., Ye, K., Fazzari, M. J., Suzuki, M., Stasiek, E., Figueroa, M. E., Glass, J. L., Chen, Q., Montagna, C., et al. 2006. Comparative isoschizomer profiling of cytosine methylation: the HELP assay. Genome Res 16:1046-1055.
23. Figueroa, M. E., Reimers, M., Thompson, R. F., Ye, K., Li, Y., Selzer, R. R., Fridriksson, J., Paietta, E., Wiernik, P., Green, R. D., et al. 2008. An integrative genomic and epigenomic approach for the study of transcriptional regulation. PLoS ONE 3:e1882.
24. Oda, M., Glass, J. L., Thompson, R. F., Mo, Y., Olivier, E. N., Figueroa, M. E., Selzer, R. R., Richmond, T. A., Zhang, X., Dannenberg, L., et al. 2009. High-resolution genome-wide cytosine methylation profiling with simultaneous copy number analysis and optimization for limited cell numbers. Nucleic Acids Res 37:3829-3839.
25. Will, B., Zhou, L., Vogler, T. O., Ben-Neriah, S., Schinke, C., Tamari, R., Yu, Y., Bhagat, T. D., Bhattacharyya, S., Barreyro, L., et al. 2012. Stem and progenitor cells in myelodysplastic syndromes show aberrant stage-specific expansion and harbor genetic and epigenetic alterations. Blood 120:2076-2086.
26. Heuck, C. J., Mehta, J., Bhagat, T., Gundabolu, K., Yu, Y., Khan, S., Chrysofakis, G., Schinke, C., Tariman, J., Vickrey, E., et al. 2013. Myeloma is characterized by stage-specific alterations in DNA methylation that occur early during myelomagenesis. J Immunol 190:2966-2975.
27. Behre, G., Singh, S. M., Liu, H., Bortolin, L. T., Christopeit, M., Radomska, H. S., Rangatia, J., Hiddemann, W., Friedman, A. D., and Tenen, D. G. 2002. Ras signaling enhances the activity of C/EBP alpha to induce granulocytic differentiation by phosphorylation of serine 248. J Biol Chem 277:26293-26299.
28. Geletu, M., Balkhi, M. Y., Peer Zada, A. A., Christopeit, M., Pulikkan, J. A., Trivedi, A. K., Tenen, D. G., and Behre, G. 2007. Target proteins of C/EBPalphap30 in AML: C/EBPalphap30 enhances sumoylation of C/EBPalphap42 via up-regulation of Ubc9. Blood 110:3301-3309.
29. Pulikkan, J. A., Peramangalam, P. S., Dengler, V., Ho, P. A., Preudhomme, C., Meshinchi, S., Christopeit, M., Nibourel, O., Miiller-Tidow, C., Bohlander, S. K., et al. 2010. C/EBPalpha regulated microRNA-34a targets E2F3 during granulopoiesis and is down-regulated in AML with CEBPA mutations. Blood 116:5638-5649.
30. Pulikkan, J. A., Dengler, V., Peramangalam, P. S., Peer Zada, A. A., Muller-Tidow, C., Bohlander, S. K., Tenen, D. G., and Behre, G. 2010. Cell-cycle regulator E2F1 and microRNA-223 comprise an autoregulatory negative feedback loop in acute myeloid leukemia. Blood 115:1768-1778.
31. Braun, B. S., Archard, J. A., Van Ziffle, J. A., Tuveson, D. A., Jacks, T. E., and Shannon, K. 2006. Somatic activation of a conditional KrasG12D allele causes ineffective erythropoiesis in vivo. Blood 108:2041-2044.
32. Zhang, J., Ding, L., Holmfeldt, L., Wu, G., Heatley, S. L., Payne-Turner, D., Easton, J., Chen, X., Wang, J., Rusch, M., et al. 2012. The genetic basis of early T-cell precursor acute lymphoblastic leukaemia. Nature 481:157-163.
33. Porter, C. C., Kim, J., Fosmire, S., Gearheart, C. M., van Linden, A., Baturin, D., Zaberezhnyy, V., Patel, P. R., Gao, D., Tan, A. C., et al. 2012. Integrated genomic analyses identify WEE1 as a critical mediator of cell fate and a novel therapeutic target in acute myeloid leukemia. Leukemia 26:1266-1276.
34. Sun, S. M., Dijkstra, M. K., Bijkerk, A. C., Brooimans, R. A., Valk, P. J., Erkeland, S. J., Lowenberg, B., and Jongen-Lavrencic, M. 2011. Transition of highly specific microRNA expression patterns in association with discrete maturation stages of human granulopoiesis. Br J Haematol 155:395-398.

35. Garzon, R., and Croce, C. M. 2008. MicroRNAs in normal and malignant hematopoiesis. Curr Opin Hematol 15:352-358.
36. Chen, J., Odenike, O., and Rowley, J. D. 2010. Leukaemogenesis: more than mutant genes. Nat Rev Cancer 10:23-36.
37. Jiang, X., Huang, H., Li, Z., Li, Y., Wang, X., Gurbuxani, S., Chen, P., He, C., You, D., Zhang, S., et al. 2012. Blockade of miR-150 maturation by MLL-fusion/MYC/LIN-28 is required for MLL-associated leukemia. Cancer Cell 22:524-535.
38. Li, Z., Huang, H., Chen, P., He, M., Li, Y., Arnovitz, S., Jiang, X., He, C., Hyjek, E., Zhang, J., et al. 2012. miR-196b directly targets both HOXA9/MEIS1 oncogenes and FAS tumour suppressor in MLL-rearranged leukaemia. Nat Commun 3:688.
39. Valk, P. J., Verhaak, R. G., Beijen, M. A., Erpelinck, C. A., Barjesteh van Waalwijk van Doorn-Khosrovani, S., Boer, J. M., Beverloo, H. B., Moorhouse, M. J., van der Spek, P. J., Lowenberg, B., et al. 2004. Prognostically useful gene-expression profiles in acute myeloid leukemia. N Engl J Med 350:1617-1628.
40. Verhaak, R. G., Wouters, B. J., Erpelinck, C. A., Abbas, S., Beverloo, H. B., Lugthart, S., Lowenberg, B., Delwel, R., and Valk, P. J. 2009. Prediction of molecular subtypes in acute myeloid leukemia based on gene expression profiling. Haematologica 94:131-134.
41. Fernandez, H. F., Sun, Z., Yao, X., Litzow, M. R., Luger, S. M., Paietta, E. M., Racevskis, J., Dewald, G. W., Ketterling, R. P., Bennett, J. M., et al. 2009. Anthracycline dose intensification in acute myeloid leukemia. N Engl J Med 361:1249-1259.
42. Figueroa, M. E., Abdel-Wahab, O., Lu, C., Ward, P. S., Patel, J., Shih, A., Li, Y., Bhagwat, N., Vasanthakumar, A., Fernandez, H. F., et al. 2010. Leukemic IDH1 and IDH2 mutations result in a hypermethylation phenotype, disrupt TET2 function, and impair hematopoietic differentiation. Cancer Cell 18:553-567.
43. Goeman, J. J., van de Geer, S. A., de Kort, F., and van Houwelingen, H. C. 2004. A global test for groups of genes: testing association with a clinical outcome. Bioinformatics 20:93-99.
44. Cox, D. R. 1972. Regression models and life tables. J R Stat Soc B 34:187-220.
45. Gentles, A. J., Plevritis, S. K., Majeti, R., and Alizadeh, A. A. 2010. Association of a leukemic stem cell gene expression signature with clinical outcomes in acute myeloid leukemia. JAMA 304:2706-2715.
46. Eppert, K., Takenaka, K., Lechman, E. R., Waldron, L., Nilsson, B., van Galen, P., Metzeler, K. H., Poeppl, A., Ling, V., Beyene, J., et al. 2011. Stem cell gene expression programs influence clinical outcome in human leukemia. Nat Med 17:1086-1093.
47. Li, Z., Herold, T., He, C., Valk, P. J., Chen, P., Jurinovic, V., Mansmann, U., Radmacher, M. D., Maharry, K. S., Sun, M., et al. 2013. Identification of a 24-gene prognostic signature that improves the European LeukemiaNet risk classification of acute myeloid leukemia: an international collaborative study. J Clin Oncol 31:1172-1181.
48. Deaton, A. M., and Bird, A. 2011. CpG islands and the regulation of transcription. Genes Dev 25:1010-1022.
49. Suzuki, M., Oda, M., Ramos, M. P., Pascual, M., Lau, K., Stasiek, E., Agyiri, F., Thompson, R. F., Glass, J. L., Jing, Q., et al. 2011. Late-replicating heterochromatin is characterized by decreased cytosine methylation in the human genome. Genome Res 21:1833-1840.
50. Heinz, S., Benner, C., Spann, N., Bertolino, E., Lin, Y. C., Laslo, P., Cheng, J. X., Murre, C., Singh, H., and Glass, C. K. 2010. Simple combinations of lineage-determining transcription factors prime cis-regulatory elements required for macrophage and B cell identities. Mol Cell 38:576-589.
51. Bhagat, T. D., Zhou, L., Sokol, L., Kessel, R., Caceres, G., Gundabolu, K., Tamari, R., Gordon, S., Mantzaris, I., Jodlowski, T., et al. 2013. miR-21 mediates hematopoietic suppression in MDS by activating TGF-beta signaling. Blood 121:2875-2881.
52. Zhou, L., McMahon, C., Bhagat, T., Alencar, C., Yu, Y., Fazzari, M., Sohal, D., Heuck, C., Gundabolu, K., Ng, C., et al. 2011. Reduced SMAD7 leads to overactivation of TGF-beta signaling in MDS that can be reversed by a specific inhibitor of TGF-beta receptor I kinase. Cancer Res 71:955-963.
53. Zhou, L., Nguyen, A. N., Sohal, D., Ying Ma, J., Pahanish, P., Gundabolu, K., Hayman, J., Chubak, A., Mo, Y., Bhagat, T. D., et al. 2008 Inhibition of the TGF-beta receptor I kinase promotes hematopoiesis in MDS. Blood 112:3434-3443.
54. Schubeler, D. 2012. Molecular biology. Epigenetic islands in a genetic ocean. Science 338:756-757.
55. Yu, Y., Mo, Y., Ebenezer, D., Bhattacharyya, S., Liu, H., Sundaravel, S., Giricz, O., Wontakal, S., Cartier, J., Caces, B., et al. 2013. High resolution methylome analysis reveals widespread functional hypomethylation during adult human erythropoiesis. J Biol Chem 288:8805-8814.
56. Gonen, M., Sun, Z., Figueroa, M. E., Patel, J. P., Abdel-Wahab, O., Racevskis, J., Ketterling, R. P., Fernandez, H., Rowe, J. M., Tallman, M. S., et al. 2012. CD25 expression status improves prognostic risk classification in AML independent of established biomarkers: ECOG phase 3 trial, E1900. Blood 120:2297-2306.
57. Zhou, L., Opalinska, J., Sohal, D., Yu, Y., Mo, Y., Bhagat, T., Abdel-Wahab, O., Fazzari, M., Figueroa, M., Alencar, C., et al. 2011. Aberrant epigenetic and genetic marks are seen in myelodysplastic leukocytes and reveal Dock4 as a candidate pathogenic gene on chromosome 7q. J Biol Chem 286:25211-25223.
58. Nischal, S., Bhattacharyya, S., Christopeit, M., Yu, Y., Zhou, L., Bhagat, T. D., Sohal, D., Will, B., Mo, Y., Suzuki, M., et al. 2013. Methylome profiling reveals distinct alterations in phenotypic and mutational subgroups of myeloproliferative neoplasms. Cancer Res 73:1076-1085.
59. Figueroa, M. E., Chen, S. C., Andersson, A. K., Phillips, L. A., Li, Y., Sotzen, J., Kundu, M., Downing, J. R., Melnick, A., and Mullighan, C. G. 2013. Integrated genetic and epigenetic analysis of childhood acute lymphoblastic leukemia. J Clin Invest 123:3099-3111.
60. Figueroa, M. E., Wouters, B. J., Skrabanek, L., Glass, J., Li, Y., Erpelinck-Verschueren, C. A., Langerak, A. W., Lowenberg, B., Fazzari, M., Greally, J. M., et al. 2009. Genome-wide epigenetic analysis delineates a biologically distinct immature acute leukemia with myeloid/T-lymphoid features. Blood 113:2795-2804.
61. Thompson, R. F., Reimers, M., Khulan, B., Gissot, M., Richmond, T. A., Chen, Q., Zheng, X., Kim, K., and Greally, J. M. 2008. An analytical pipeline for genomic representations used for cytosine methylation studies. Bioinformatics 24:1161-1167.
62. Kaplan, E. L., and Meier, P. 1958. Nonparametric Estimation from Incomplete Observations. Journal of the American Statistical Association 53:457-481.
63. Kawahara, M., Pandolfi, A., Bartholdy, B., Barreyro, L., Will, B., Roth, M., Okoye-Okafor, U. C., Todorova, T. I., Figueroa, M. E., Melnick, A., et al. 2012. H2.0-like Homeobox Regulates Early Hematopoiesis and Promotes Acute Myeloid Leukemia. Cancer Cell 22:194-208.

1. A method for determining the presence of gene methylation above or below a predetermined amount in a subject having acute myeloid leukemia (AML), comprising
a) quantifying the methylation of DNA of a sample comprising blood cells or bone marrow cells obtained from a subject with AML at a plurality of chromosome loci, or nearest associated gene, as listed in Table 2;
b) determining a methylation score from the methylation determined in step a);
c) comparing the methylation score of DNA of the sample of blood cells obtained from the subject with AML with a predetermined reference amount for the same plurality of chromosome loci, or nearest associated gene, and
d) assigning a level of methylation to the subject,
wherein a methylation score at or in excess of the predetermined reference amount indicates a negative AML prognosis for the subject,
and wherein a methylation score below the predetermined reference amount indicates a positive prognosis AML for the subject.

2. The method of claim 1, wherein the methylation score comprises a direct or indirect measurement of the ratio of demethylated CpG residues/methylated+demethylated CpG residues of the plurality of the chromosome loci, or nearest associated gene for the DNA of a sample.

3. The method of claim 1, wherein the methylation is determined by a isoschizomer enzyme pair method, and wherein the methylation score is obtained by summing absolute values of the median-centered methylation values (log 2[methylation sensitive enzyme measured fragments/methylation insensitive enzyme measured fragments]) of the plurality of the chromosome loci, or nearest associated gene for the DNA of a sample.

4. The method of claim 1, wherein the isoschizomer enzyme pair is HpaII and MspI.

5. The method of claim 1, wherein the HELP assay is used to determine the methylation of the DNA.

6. A method for determining the prognosis of a subject having acute myeloid leukemia (AML), comprising
quantifying methylation of DNA of a sample comprising blood cells obtained from a subject with AML at a plurality of the chromosome loci, or nearest associated gene, listed in Table 2 as demethylated at STHSC-CMP transition and/or at a plurality of the chromosome loci, or nearest associated gene, listed in Table 2 as methylated at STHSC-CMP transition,
comparing the extent of the methylation with the methylation of DNA at the same plurality or pluralities of chromosome loci, or nearest associated gene, in a sample of blood cells obtained from a subject without AML, and
assigning a prognosis to the subject,
wherein demethylation of the majority of the plurality of loci or nearest associated gene listed in Table 2 as demethylated at STHSC-CMP transition in the DNA of the sample from the AML subject compared to the DNA of the sample of the subject without AML indicates a negative prognosis for the subject, and wherein methylation of the majority of the plurality of loci or nearest associated gene listed in Table 2 as methylated at STHSC-CMP transition in the DNA of the sample from the AML subject compared to the DNA of the sample from the subject without AML indicates a negative prognosis for the subject,
and wherein demethylation of the minority of the plurality of loci or nearest associated gene listed in Table 2 as demethylated at STHSC-CMP transition in the DNA of the sample from the AML subject compared to the DNA of the sample of the subject without AML indicates a negative prognosis for the subject, and wherein methylation of the minority of the plurality of loci or nearest associated gene listed in Table 2 as methylated at STHSC-CMP transition in the DNA of the sample from the AML subject compared to the DNA of the sample from the subject without AML indicates a negative prognosis for the subject.

7. The method of claim 1, wherein quantifying methylation is effected by recovering DNA from the blood cells digesting a first portion of the DNA with a methylation-sensitive restriction enzyme and a second portion of the DNA with a methylation-insensitive restriction enzyme, and hybridizing to a HELP microarray.

8. The method of claim 1, wherein quantifying methylation is effected using HpaII tiny fragment Enrichment by Ligation-mediated PCR.

9. The method of claim 1, wherein quantifying methylation is effected by contacting a first portion of the DNA with sodium bisulfite under conditions permitting conversion of cytosine residues of the DNA into uracils, sequencing the DNA of the first portion and of a second portion untreated with sodium bisulfite, and aligning the resultant sequences of the two portions and comparing the sequences so as to determine the extent and position of methylated nucleotides in the DNA.

10. The method of claim 9, further comprising PCR amplifying the DNA after contacting with sodium bisulfite but prior to sequencing.

11. The method of claim 1, wherein the plurality of loci or nearest associated gene listed in Table 2 comprises at least 5 loci or nearest associated genes.

12. The method of claim 1, wherein the plurality of loci or nearest associated gene listed in Table 2 comprises at least 10 loci or nearest associated genes.

13. The method of claim 1, wherein the plurality of loci or nearest associated gene listed in Table 2 comprises at least 100 loci or nearest associated genes.

14. (canceled)

15. The method of claim 6, wherein the plurality of loci or nearest associated gene listed in Table 2 as demethylated at STHSC-CMP transition comprises at least 5 loci or nearest associated genes.

16-22. (canceled)

23. The method of claim 1, wherein the methylation is quantified as DNA cytosine methylation.

24. A method for treating a subject having acute myeloid leukemia (AML) comprising:
a) receiving identification of the subject as having a positive or negative prognosis by the method of claim 1; and
b) treating the subject with a chemotherapy if the subject has a positive prognosis or treating the subject with a non-chemotherapeutic method if the subject has a negative prognosis.

25. The method of claim 24, wherein the chemotherapy comprises administering an anthracycline and/or cytarabine and/or a demethylating agent, and or/a TKI.

26. The method of claim 25, wherein the anthracycline is daunorubicin.

27. The method of claim 25, wherein the non-chemotherapeutic method comprises an allogeneic stem cell transplantation into the subject.

28. A kit for determining the prognosis of a subject having acute myeloid leukemia (AML), comprising
a) reagents for quantifying the methylation of DNA of a sample comprising blood cells or bone marrow cells obtained from a subject with AML at a plurality of chromosome loci, or nearest associated gene, as listed in Table 2;
b) written instructions for determining a methylation score from the methylation determined with the reagents in a);
c) written instructions for a predetermined reference amount for the same plurality of chromosome loci, or nearest associated gene, and for assigning a prognosis to the subject based on the methylation score compared to the predetermined reference amount,
wherein a methylation score at or in excess of the predetermined reference amount indicates a negative prognosis for the subject,
and wherein a methylation score below the predetermined reference amount indicates a positive prognosis for the subject.

29-32. (canceled)
